(19) United States
(12) Patent Application Publication
(10) Pub. No.: US 2015/0133989 A1
(43) Pub. Date: May 14, 2015

(54) EXPANDABLE OCCLUSION DEVICES AND METHODS OF USE

(71) Applicant: Inceptus Medical, LLC, Aliso Viejo, CA (US)

(72) Inventors: Paul Lubock, Monarch Beach, CA (US); Brian J. Cox, Laguna Niguel, CA (US); Robert Rosenbluth, Laguna Niguel, CA (US); Richard Quick, Mission Viejo, CA (US)

(21) Appl. No.: 14/395,783

(22) PCT Filed: Apr. 19, 2013
(86) PCT No.: PCT/US13/37484

§ 371 Date: Oct. 20, 2014

Related U.S. Application Data
(60) Provisional application No. 61/636,392, filed on Apr. 20, 2012.

(30) Foreign Application Priority Data
Aug. 17, 2012 (US) PCT/US12/51502
Jan. 4, 2013 (US) PCT/US13/20381

(51) Int. Cl. A61B 17/12

(52) U.S. Cl. CPC A61B 17/12172; A61B 17/12031; A61B 17/12109; A61B 17/12122; A61B 2017/1205
USPC 606/200

(57) ABSTRACT

An occlusion device and method for occluding an undesirable vascular structure, such as a septal defect or left atrial appendage. The occlusion device includes a lattice structure that expands from a contracted catheter-deliverable state to an expanded state that occludes the vascular structure. The lattice structure has one or more braided layers, with structural braided layers that provide structural support to the device, and occlusive layers that provide a lattice braiding or pore sizes that promote further occlusion by a biological process, such as tissue ingrowth that further occludes the affected vascular structure.

### CROSS-REFERENCE TO RELATED APPLICATIONS

The present application claims the benefit of U.S. Provisional Application No. 61/636,392, filed Apr. 20, 2012, entitled “DEVICES AND METHODS FOR VASCULAR OCCLUSION,” PCT Application No. PCT/US12/51502, filed Aug. 17, 2012, entitled “EXPANDABLE OCCLUSION DEVICES AND METHODS,” and PCT Application No. PCT/US13/20381, filed Jan. 4, 2013, entitled “EXPANDABLE OCCLUSION DEVICES AND METHODS OF USE,” the full disclosures of which are incorporated herein by reference.

### TECHNICAL FIELD

The present technology relates generally to cardiovascular devices, implant delivery systems, and methods of using cardiovascular devices and delivery systems to treat structural and functional defects in the heart and circulatory system. More specifically, the present technology is directed to the occlusion of undesirable blood flow passages to repair or mitigate structural heart defects and/or diminished blood flow characteristics.

### BACKGROUND

The human cardiovascular system is composed of the heart, blood, and blood vessels. As shown in FIG. 1, the heart (H) is a muscular organ that has four main chambers: the right atrium (RA), the left atrium (LA), the right ventricle (RV), and the left ventricle (LV). The right atrium (RA) and the right ventricle (RV) are separated by a muscular wall or septum (S) from the left atrium (LA) and the left ventricle (LV), respectively. During normal systemic circulation, veins carry deoxygenated blood from the body to the right atrium (RA). The right ventricle (RV) receives the deoxygenated blood from the right atrium (RA) which is then pumped to the lungs through the pulmonary artery (PA). Oxygenated blood returns from the lungs at the left atrium (LA) and is pumped to the left ventricle (LV), which then distributes the oxygen-rich blood to the body via the aorta (A) and the peripheral arteries.

Congenital heart disorders such as patent ductus arteriosus (PDA), atrial septal defects (ASD), and ventricular septal defects (VSD) can result in abnormal openings between the walls of the heart and/or nearby blood vessels. During pregnancy, oxygenated blood is supplied by the mother to the fetus and consequently, small openings are present in the fetal heart and major vessels to bypass the pulmonary circulation. In a newborn having a congenital heart defect, however, these openings or other similar formations fail to close properly. For example, a patent ductus arteriosus (PDA) is a congenital defect wherein the ductus arteriosus, a normal fetal blood vessel connecting the aorta (A) and the pulmonary artery (PA), fails to close during neonatal development (FIG. 2). Septal defects are another form of congenital disorders involving an abnormal opening in the septum (S) that allows an undesirable net flow of blood that deviates from the directional systemic circulation described above (e.g., shunting). For example, FIG. 3 shows an opening in the septum (S) between the left atrium (LA) and right atrium (RA) generally known as an atrial septal defect (ASD). One common form of ASD is a patent foramen ovale (PFO) which forms when a flap of tissue across the atrial septal opening (the foramen ovale) does not fuse shut during neonatal development. Opening(s) in the septum (S) between the right ventricle (RV) and the left ventricle (LV) are known as ventricular septal defects (VSD) (FIG. 4).

The congenital heart defects described above can cause cardiac and related problems including congestive heart failure, pulmonary hypertension, cryptogenic stroke, transient ischemic attack (TIA), clots, emboli, migraines, and others. As a result, in some cases it may be necessary to partially or fully occlude the abnormal opening or vessel to stop the undesired blood flow.

In addition to congenital heart defects, other abnormal openings and/or undesirable blood flow in the body's vasculature can also necessitate medical treatment to fully or partially occlude the vessel and/or body cavity. Undesired blood flow can include blood flow to certain body cavities, tumors, fistulas, aneurysms and others. For example, the lateral wall of the left atrium (LA) has a muscular pouch or cavity known as the left atrial appendage (“LAA”) (see FIG. 5). Although the exact function of the LAA is not known, during normal left atrial filling the LAA also fills and blood is expelled with the contraction of the left atrium (LA). In some disease states, particularly a condition known as atrial fibrillation, the contraction of the LAA may be inhibited or inconsistent and pooling of blood in the LAA may occur. The pooled blood may clot and subsequently embolize into the arterial circulation potentially leading to embolic stroke of the brain, heart or other vital organs.

Minimally invasive approaches to cardiac and/or vascular occlusion have been developed in recent years, such as transcatheter occlusion devices. These devices, however, have drawbacks such as insufficient tissue sealing, inadequate fixation of the device at the target location, poor hemodynamic design leading to excessive thrombus formation, and other drawbacks described in more detail below. Accordingly, there is a need for devices and methods that address one or more of these deficiencies.

### BRIEF DESCRIPTION OF THE DRAWINGS

The accompanying drawings, which are incorporated herein and constitute part of this specification, illustrate embodiments of the present technology, and, together with the general description given above and the detailed description given below, serve to explain the features of the present technology.

FIG. 1 is a schematic cross-sectional view of a normal heart.

FIG. 2 is a schematic cross-sectional view of a heart showing a patent ductus arteriosus.

FIG. 3 is a schematic cross-sectional view of a heart showing an atrial septal defect.

FIG. 4 is a schematic cross-sectional view of a heart showing a ventricular septal defect.

FIG. 5 is a posteroinferior view of a heart showing the left atrial appendage.

FIG. 6A is a side view of an occlusion device for placement within a vascular structure of the body in accordance with an embodiment of the present technology.

FIG. 6B is a cross-sectional side view of the expandable occlusion device of FIG. 6A configured in accordance with an embodiment of the present technology.

FIG. 6C is an enlarged view of a proximal hub of FIG. 6A configured in accordance with embodiments of the present technology.

FIG. 6D is an enlarged view of the outer distal hub of FIG. 6A configured in accordance with embodiments of the present technology.

FIG. 6E is a side view of an occlusion device comprising a single layer occlusive braid in accordance with embodiments of the present technology.

FIG. 6F is a side view of an occlusion device for placement within a vascular structure of the body in accordance with an embodiment of the present technology.

FIG. 6G is a cross-sectional side view of the expandable occlusion device of FIG. 6F configured in accordance with an embodiment of the present technology.

FIG. 6H is a cross-sectional side view of an occlusion device for placement within or at a septal defect configured in accordance with an embodiment of the present technology.

FIG. 6I is a cross-sectional side view of an occlusion device for placement within or at a septal defect configured in accordance with another embodiment of the present technology.

FIG. 6J is an anatomical side view of an expandable occlusion device positioned at a PFO configured in accordance with an embodiment of the present technology.

FIG. 6K is an anatomical side view of an expandable occlusion device positioned at a PFO configured in accordance with an embodiment of the present technology.

FIG. 7A is a perspective view of an expanded occlusion device having retention members in accordance with an embodiment of the present technology.

FIG. 7B is an enlarged cross-sectional view of a section of FIG. 7A in accordance with an embodiment of the present technology.

FIGS. 7C-5K show different embodiments of retention members in accordance with the present technology.

FIG. 7L is a perspective view of an expanded occlusion device having retention members in accordance with an embodiment of the present technology.

FIG. 7M is a perspective cross-sectional view of an expanded occlusion device having an outer anchoring lattice configured in accordance with an embodiment of the present technology.

FIG. 7N is an anatomical side view showing an expanded occlusion device having a terminal retention member positioned at a patent ductus arteriosus configured in accordance with an embodiment of the present technology.

FIG. 7O is a perspective view of a terminal retention member configured in accordance with an embodiment of the present technology.

FIGS. 7P-7R are schematic top views of an expanded occlusion device having a terminal retention member configured in accordance with various embodiments of the present technology.

FIG. 8A is a schematic cross-sectional view of one embodiment of a delivery system is configured in accordance with an embodiment of the present technology.

FIG. 8B is an enlarged cross-sectional side view of select components at a distal region of an occlusion device delivery system in accordance with an embodiment of the present technology.

FIG. 9A shows a typical antegrade approach to the right atrium of the heart.

FIG. 9B shows a typical antegrade approach to the left atrium of the heart.

FIG. 9C shows a typical antegrade approach to the left atrial appendage of the heart.

FIG. 9D is a side perspective view of a guidewire and delivery catheter positioned at or near a target location in a vascular structure in accordance with an embodiment of the present technology.

FIG. 9E is a side perspective view of a partially expanded occlusion device during deployment at or near a target location in a vascular structure in accordance with an embodiment of the present technology.

FIG. 9F is a side perspective view of an expandable occlusion device in a deployed state (e.g., expanded configuration) positioned at the left atrial appendage in accordance with an embodiment of the present technology.

FIG. 9G is a side perspective view of an expandable occlusion device in a deployed state (e.g., expanded configuration) positioned at an aneurysm in accordance with an embodiment of the present technology.

FIG. 10A is a schematic side view of one embodiment of a delivery system having a balloon positioning member in accordance with an embodiment of the present technology.

FIG. 10B is a schematic side view of one embodiment of a delivery system having an expandable mesh positioning member in accordance with an embodiment of the present technology.

FIG. 10C is a schematic side view of one embodiment of a delivery system having a Malecot positioning member in accordance with an embodiment of the present technology.

FIG. 10D is a schematic side view of one embodiment of a delivery system having a mechanical positioner positioning member in accordance with an embodiment of the present technology.

FIG. 11A is a side view of a mandrel and a braided mesh formed over the mandrel configured in accordance with an embodiment of the present technology.

FIG. 11B is an enlarged view of a self-expanding braid with interwoven large and small strands configured in accordance with an embodiment of the present technology.

FIG. 11C is an enlarged view of a braid showing a pore.

FIG. 11D is an enlarged top view of an end region of an occlusion device.

FIG. 12A is a schematic side view of an occlusion device having a proximal section and a distal section in accordance with an embodiment of the present technology.

FIG. 12B is a schematic side view of an occlusion device having a proximal section with a flange in accordance with an embodiment of the present technology.

FIG. 12C is a schematic side view of an occlusion device having a proximal section, a middle section, and a distal section in accordance with an embodiment of the present technology.

FIG. 12D is a schematic side view of an occlusion device having annular sections in accordance with an embodiment of the present technology.

FIG. 12E is a schematic side view of an occlusion device having a proximal section and a distal section coupled by a spring in accordance with an embodiment of the present technology.

FIG. 12F is a schematic side view of an occlusion device having a mechanically coupled proximal section and distal section in accordance with an embodiment of the present technology.

FIG. 13A is a schematic cross-sectional side view of an occlusion device having nested sections, in accordance with an embodiment of the present technology.

FIG. 13B is a schematic side view of the occlusion device of FIG. 13A when stretched, in accordance with an embodiment of the present technology.

FIG. 14A is cross-sectional side view of an occlusion device including at least one braided layer having a free end configured in accordance with an embodiment of the present technology.

FIG. 14B is cross-sectional anatomical side view of an expanded occlusion device including at least one braided layer having a free end, positioned in a blood vessel, configured in accordance with the present technology.

FIGS. 15A-15B are cross-sectional side views of an occlusion device including at least one braided layer having a free end configured in accordance with an embodiment of the present technology.

FIGS. 16A-16B are cross-sectional side views of an occlusion device having undulated contact portions configured in accordance with an embodiment of the present technology.

FIG. 17A is a cross-sectional side view of an occlusion device having substantially closed ring volumes configured in accordance with an embodiment of the present technology.

FIG. 17B is a cross-sectional side view of an occlusion device configured in accordance with an embodiment of the present technology.

FIG. 17C is a cross-sectional side view of an occlusion device having a ringed pocket configured in accordance with an embodiment of the present technology.

FIG. 18 is a cross-sectional side view of an occlusion device having an outer layer, an intermediate layer, and an inner layer, configured in accordance with an embodiment of the present technology.

FIG. 19A is a schematic illustration showing a braid being placed over a mandrel for partial heat setting.

FIG. 19B is a conceptual illustration showing a partial heat setting process.

FIG. 19C is a schematic illustration showing eversion of one end of a braid configured in accordance with the present technology.

FIG. 19D is a cross-sectional side view of an everted braid configured in accordance with an embodiment of the present technology.

FIG. 20A is a side view of a mandrel and a braided mesh formed over the mandrel configured in accordance with an embodiment of the present technology.

FIG. 20B is a side view of a braid in an “as-braided” configuration in accordance with an embodiment of the present technology.

FIG. 20C is a side view of an expanded mold and a contracted mold configured in accordance with an embodiment of the present technology.

FIG. 20D is a conceptual illustration showing a portioned heat setting process.

FIG. 20E is a cross-sectional side view of an everted braid configured in accordance with an embodiment of the present technology.

FIGS. 21A-21C are various embodiments of spherical-shaped occlusion devices configured in accordance with embodiments of the present technology.

FIGS. 22A-22C are various embodiments of barrel-shaped occlusion devices configured in accordance with embodiments of the present technology.

FIGS. 23A-23C are various embodiments of frustum-shaped occlusion devices configured in accordance with embodiments of the present technology.

### DETAILED DESCRIPTION

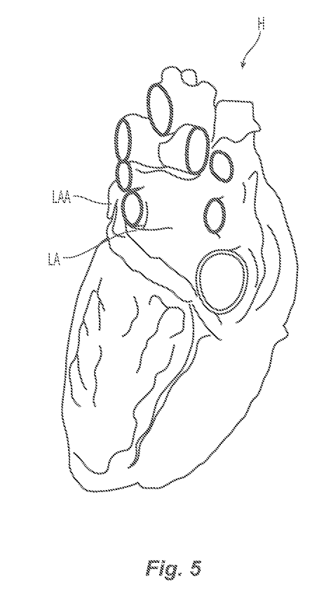

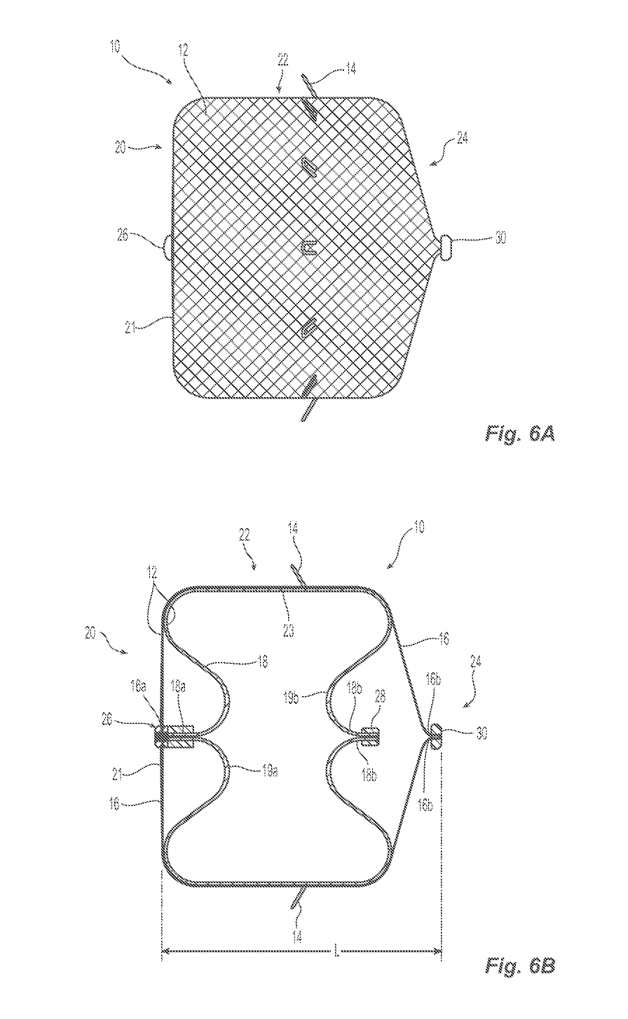

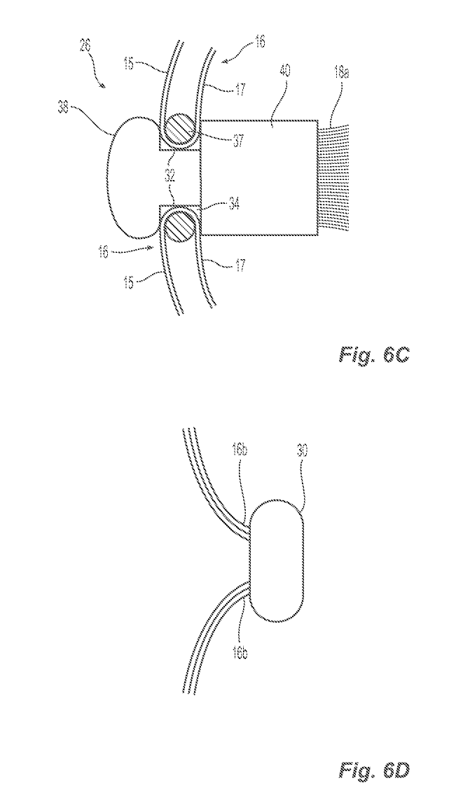

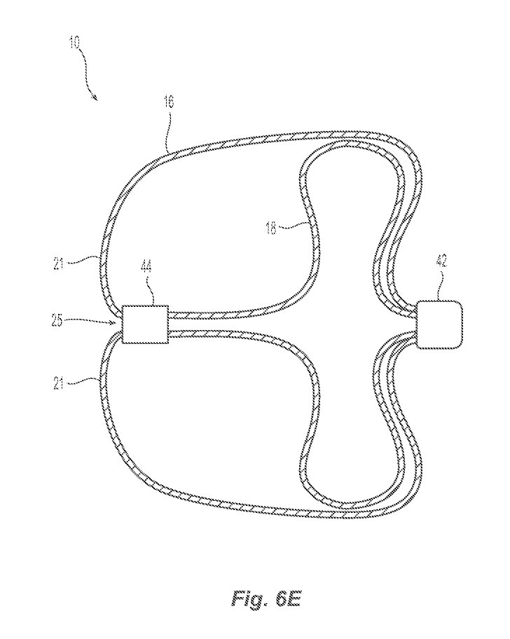

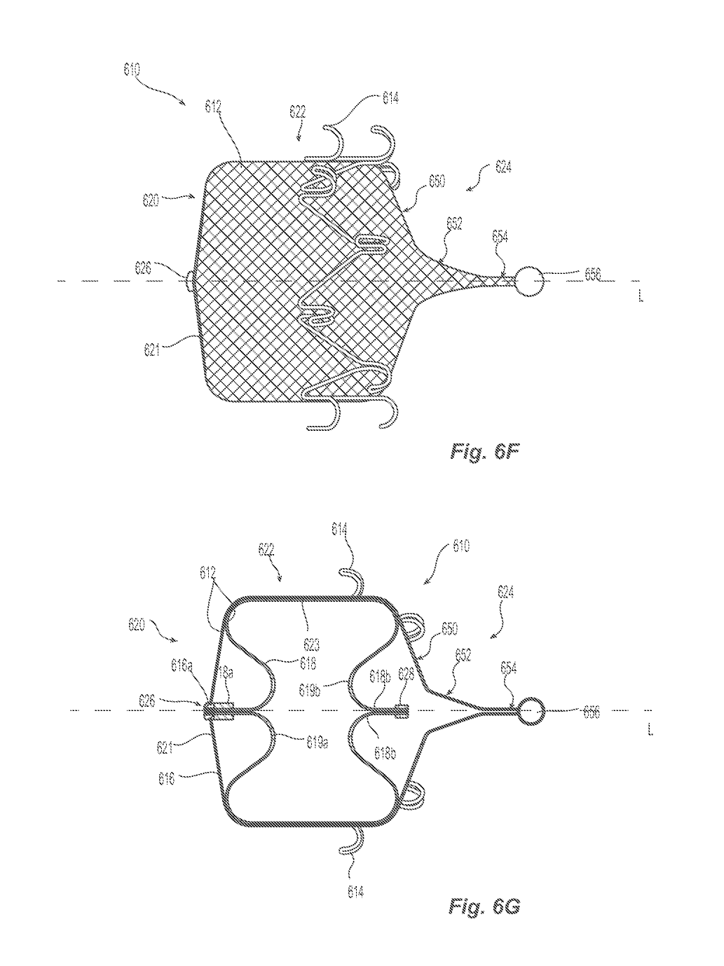

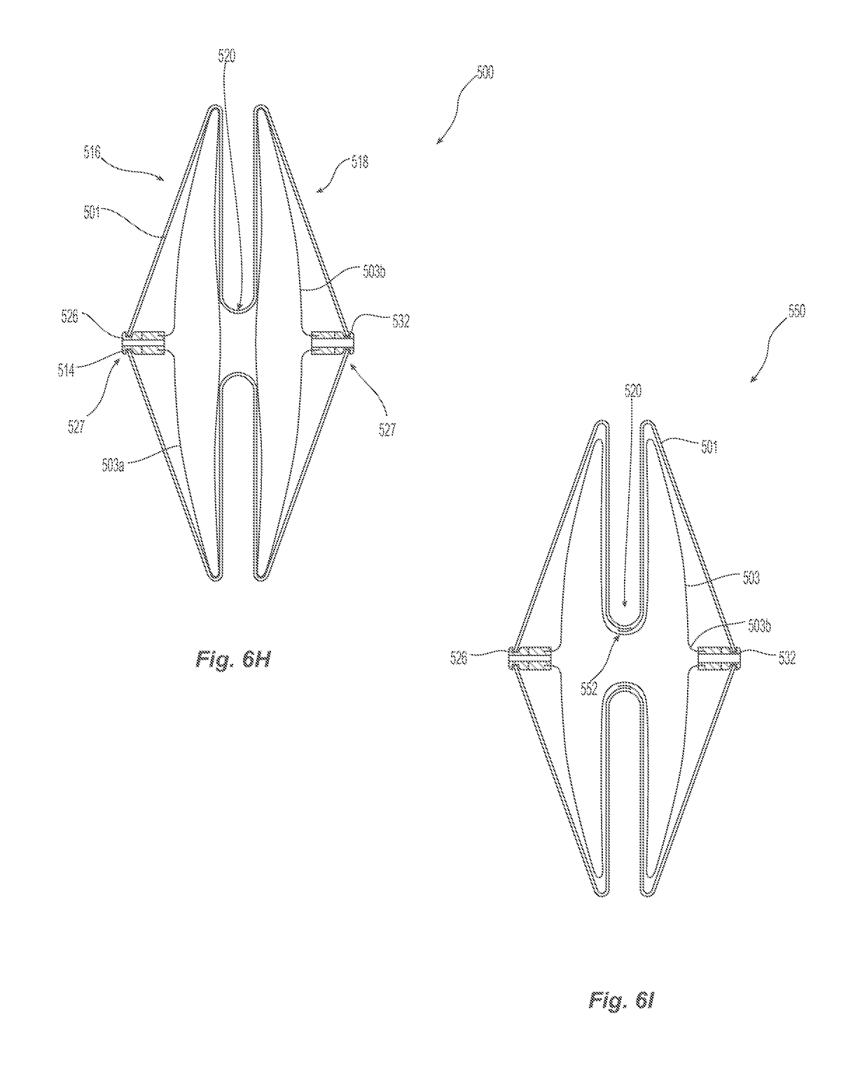

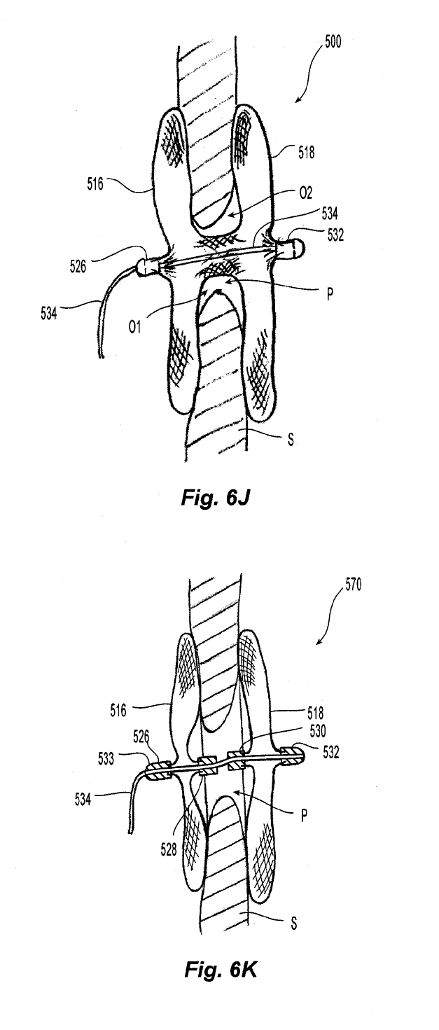

Specific details of several embodiments of the technology are described below with reference to FIGS. 6A-23C. Although many of the embodiments are described below with respect to devices, systems, and methods for occluding vascular structures (e.g., passageways and/or cavities), other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 6A-23C.

With regard to the terms “distal” and “proximal” within this description, unless otherwise specified, the terms can reference a relative position of the portions of an occlusion device and/or an associated delivery device with reference to an operator and/or a location in the vasculature. For example, proximal can refer to a position closer to the operator of the device or an incision into the vasculature, and distal can refer to a position that is more distant from the operator of the device or further from the incision along the vasculature.

For ease of reference, throughout this disclosure identical reference numbers are used to identify similar or analogous components or features, but the use of the same reference number does not imply that the parts should be construed to be identical. Indeed, in many examples described herein, identically numbered parts of individual embodiments are distinct in structure and/or function. The headings provided herein are for convenience only.

### 1. Selected Embodiments of Occlusion Devices

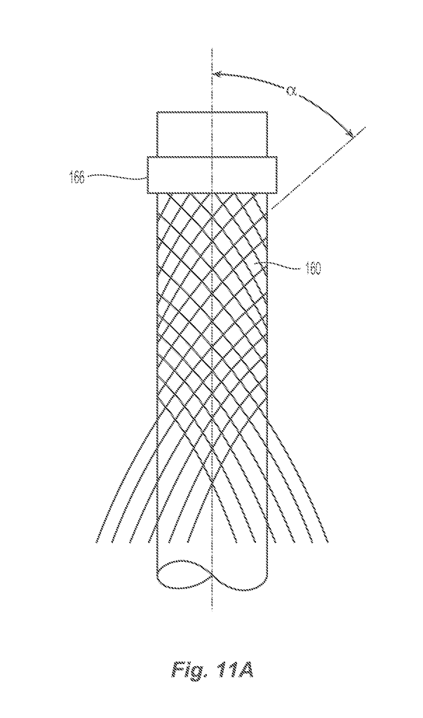

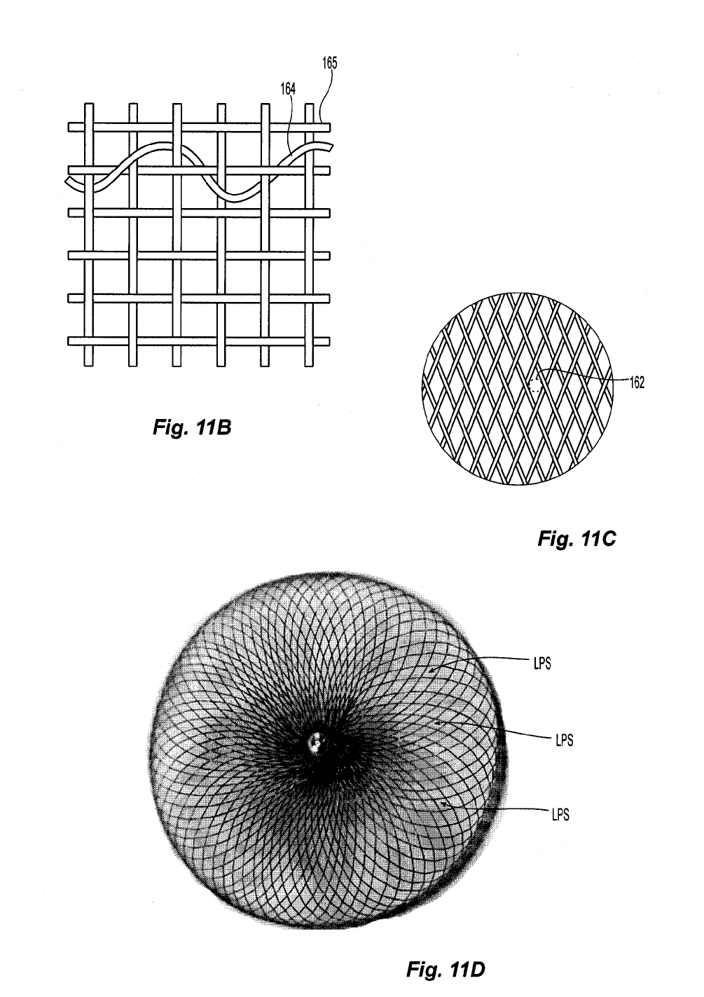

Introductory examples of occlusion devices, systems and associated methods in accordance with embodiments of the present technology are described in this section with reference to FIGS. 6A-10D. It will be appreciated that specific elements, substructures, advantages, uses, and/or other features of the embodiments described with reference to FIGS. 6A-10D can be suitably interchanged, substituted or otherwise configured with one another and/or with the embodiments described with reference to FIGS. 11A-23C in accordance with additional embodiments of the present technology. Furthermore, suitable elements of the embodiments described with reference to FIGS. 6A-23C can be used as standalone and/or self-contained devices.

Several embodiments of occlusion devices and delivery systems described herein are directed to self-expanding occlusion devices that are implanted at a location where there is an undesirable passage within tissue, such as a blood flow passage extending into cardiac or vascular tissue. A “vascular structure” as used herein includes an accessible opening within or through tissue, such as a two-ended passage connecting two portions of the cardiovascular system (e.g., a passage through a septum), a cavity, cul-de-sac and/or one-ended passage terminating within tissue (e.g., an LAA or an aneurysm), a passage exiting the cardiovascular system (e.g., a hemorrhage site), and/or an anatomical passage (e.g., a blood vessel, or a channel or duct of an organ). As described below, the occlusion device can occlude or at least partially occlude an undesired vascular structure, and the structure and shape of the occlusion device can have multiple layers of at least one self-expanding lattice structure that controls the occlusion of the vascular structure.

FIGS. 6A-6D show one embodiment of an occlusion device 10 in an unrestricted expanded configuration. As shown in the side view of FIG. 6A, the occlusion device 10 includes a flexible, self-expanding lattice structure 12 and one or more retention members 14 coupled to and/or integrated with the lattice structure 12. The lattice structure 12 can be generally cylindrical, as shown in FIG. 6A. In other embodiments, the lattice structure 12 can have a shape that is generally spherical, ellipsoidal, oval, barrel-like, conical, frustum-shaped, or any other suitable shape. The lattice structure 12 can have a proximal region 20 having a low-profile proximal face 21, a distal region 24, and a contact region 22 in between. As shown in FIG. 6A, in some embodiments the proximal face 21 can be planar or substantially planar with a slight proximal and/or distal bow, the contact region 22 can be generally cylindrical, and the distal region 24 can be tapered. The contact region 22 can provide a sufficient outward radial force to deform the vascular structure to a certain extent while also being sufficiently flexible to conform to the vascular structure such that the contact region becomes at least substantially sealed to the vascular structure tissue.

The lattice structure 12 can include one or more layers, and each layer can comprise an expandable lattice and/or a braided mesh of filaments (e.g., wires, threads, sutures, fibers, etc.). For example, as shown in the cross-sectional side view of FIG. 6B, the lattice structure 12 can include an occlusive braid 16 and a structural braid 18 arranged so that the occlusive braid 16 envelops the structural braid 18. In the illustrated embodiment shown in FIG. 6B, both the occlusive braid 16 and the structural braid 18 have proximal ends 16a and 18a, respectively, secured to a proximal hub 26, such as a wire tied or wound around the braided filament ends, an adhesive holding the ends together, a welded fastener, solder, braze, laser weld, EDM weld, other weld material, a crimp, a thermally contracted fitting, and/or other suitable fastening elements and/or devices. The outer occlusive braid 16 has distal ends 16b secured to an outer distal hub 30 and the inner structural braid 18 has distal ends 18b secured to an inner distal hub 28. The inner distal hub 28 moves independently of the outer distal hub 30 such that the occlusive and structural braids 16 and 18 can have different lengths without causing one of the braids to bunch upon collapse for delivery because the braids can move relative to each other to accommodate compression into a contracted state.

As illustrated in FIG. 6C, a substantial portion of the proximal hub 26 is encapsulated by the occlusive braid 16. Because of this, only a small portion of the hub protrudes from the proximal face 21 such that the proximal hub 26 only has a slight or negligible effect on the profile of the proximal face 21. For example, in some embodiments, the proximal hub 26 increases the profile of the proximal face 21 by less than 2 mm in the proximal direction, or in some embodiments, by less than 1 mm. Accordingly, the proximal face 21 can include a proximal hub 26 and still maintain a low-profile contour. A low-profile proximal face 21 is important since thrombi can potentially form at or along any surface of the device that is exposed to blood flow. Many existing devices have structures at a proximal region of the device which protrude into the left atrium or other vascular structure. These protrusions increase the surface area of the device and may disrupt the blood flow (for example, in the case of the LAA, at or near an atrial chamber of the heart), thus increasing the likelihood of thrombus formation on the device. Similarly, grooves and/or pockets at a proximal region of the device present the same risk. The substantially planar proximal face 21 of the proximal region 20 mitigates this risk, as does the porous nature of the lattice structure 12. It is believed that clots formed on smooth surfaces are more likely to embolize into the bloodstream than clots formed on a porous surface. The proximal face 21 of the present invention comprises a plurality of interstices (i.e., the lattice structure) in which a thrombus or portion of a thrombus can get stuck, thus decreasing the likelihood of embolization of that thrombus.

Referring to FIG. 6D, the outer distal hub 30 can have an atraumatic shape. For example, the distal hub 30 can have a cross-sectional shape such as a sphere, an oval, an ellipse, a hemisphere with a rounded edge, a “mushroom-top” shape (see FIG. 6D), and others. The outer distal hub 30 secures the distal ends 16a of the occlusive braid and serves as an extension of the occlusion device 10 that can easily be snared should the device embolize into the left atrium during and/or after placement. Several existing devices have structures and/or extensions along the length of the device or at a distal region which can cause unnecessary trauma to the vascular structure during and/or after deployment.

Referring to FIGS. 6B-6C, the outer occlusive braid 16 can have an external layer 15 and an internal layer 17 created by everting the occlusive braid 16 around an edge 32 (FIG. 6C) at each of its proximal ends 16a. In other embodiments, the occlusive braid 16 can have more or less than two layers (as discussed below with reference to FIG. 6E). As shown in the enlarged view of the proximal hub 26 in FIG. 6C, the proximal hub 26 can have an inner portion 40 within the occlusive braid 16, a cap 38 coupled to the inner portion 40, and a groove 34 between the cap 38 and the inner portion 40. The edges 32 of the occlusive braid 16 can be received in the groove 34. For example, a clamp ring 37 can urge the edges 32 inwardly to secure the occlusive braid 16 to the proximal hub 26. The proximal ends 18a of the structural braid 18 can be secured to the inner portion 40 of the proximal hub 26. The characteristics of the occlusive braid 16 can remain constant as the braided mesh continues around the everted portion 32, or it can be formed with two or more braiding techniques so that the braiding on the inside for the internal layer 17 is different than the braiding on the outside for the external layer 15. Likewise, the braiding can change to provide differing braid angles and/or pore sizes between layers and/or along the length of the occlusive braid 16, as discussed in greater detail below with reference to FIGS. 11A-11D. For example, the maximum pore size of any pore on the proximal face 21 of the occlusive braid 16 can be less than 0.6 mm. In some embodiments, the maximum pore size of any pore on the proximal face 21 is less than 0.5 mm. Referring to FIG. 6D, the distal ends 16b of the occlusive braid 16 can be secured to the outer distal hub 30 by welding.

The mesh of the occlusive braid 16 can be configured to at least substantially, if not totally, occlude blood flow into or through the vascular structure and provide a biocompatible scaffold to promote new tissue ingrowth. The occlusive braid 16 can be made from a braided mesh of metal filaments, including nickel-titanium alloys (e.g. Nitinol), platinum, cobalt-chrome alloys, Elgiloy, stainless steel, tungsten or titanium. In some embodiments, it is desirable that the occlusive braid 16 be constructed solely from metallic materials free of any polymer materials. It is believed that the exclusion of polymer materials in some embodiments may decrease the likelihood of thrombus formation on device surfaces. It is further believed that the exclusion of polymer materials in the occlusive and/or structural braids and the sole use of metallic components can provide an occlusion device with a thinner profile that can be delivered with a small catheter as compared to devices having polymeric components. For example, the delivery catheter can be about 5F to 24F, and in some embodiments, 6F to 15F. In some embodiments, the delivery catheter can be about 8F-12F.

Some existing devices include a self-expanding frame at least partially covered at a proximal region by a permeable polymer (i.e., polyester) fabric. If the device is improperly sized and does not fully expand, the polymer fabric may loosen and/or “buckle” between the struts of the frame, much like fabric of an umbrella that has folds when not fully expanded. This can cause leakage around the device as well as create grooves for potential thrombus formation, as discussed above. Furthermore, many existing devices comprise a substantially circular cross-section while many vascular structures, such as the LAA, generally have an irregular-shaped cross-section. These devices rely on the vascular structure to adapt and conform to the device, which can also cause inadequate sealing. Although the occlusive braid 16 and structural braid 18 may be in contact along a portion of the lattice structure, the braids 16 and 18 are coupled only at a proximal region, allowing for space and free movement of the occlusive braid 16 along the length L of the lattice structure 12. The mesh of the occlusive braid 16 can be configured to have a pore size, filament diameter, weave density, and/or shape to create a highly flexible outer layer that can conform and/or generally comply to the surface of the vascular structure. For example, the occlusive braid 16 can have pore sizes (described below with reference to FIG. 11A-11D) in the range of about 0.025 mm to 2.0 mm. In some embodiments, the occlusive braid 16 can have pores size in the range of 0.025 mm to 0.300 mm, outside the range of existing devices.

The structural braid 18 can comprise the innermost layer of the lattice structure 12 and stabilizes and shapes the occlusive braid 16 and/or other layers of the lattice structure 12. When expanded, the structural braid 18 can include a generally cylindrical contact portion 23 that extends proximally along a proximal folded portion 19a and extends distally along a distal folded portion 19b. When expanded, the contact portion 23 drives the occlusive braid 16 radially outward to contact the vascular structure wall and/or protrusions on the vascular structure wall. The radial force exerted by the structural braid 18 can be substantially uniformly radial and is generally sufficient to inhibit movement, dislodgement and potential embolization of the occlusion device 10. Depending on the sizing of the lattice structure 12 and/or occlusion device 10, the vascular structure wall and/or protrusions may exert a radially compressive force on the contact portion 23 (e.g., through the occlusive braid 16). The compressive force is then distributed proximally and distally along the length L of the structural braid 18 to the folded portions 19a and 19b which can fold/bend/buckle in response. In some embodiments, the structural braid 18 has an undulating proximal and/or distal portion. Accordingly, compression of the structural braid 18 can have only a slight or negligible impact on the length L of the device. In other words, a decrease in the structural braid 18 diameter has approximately no effect on the length of the contact portion 23 or slightly shortens the length of the contact portion 23. Likewise, the longitudinal distance between the proximal hub 26 and the inner distal hub 28 remains approximately the same or slightly decreases. For example, a 20% change in the diameter of the structural braid 18 can change the length of the contact portion 23 by less than 5%, and in some embodiments, by less than 1%. In some embodiments, a 50% change in the diameter of the structural braid 18 changes the length of the contact portion 23 by less than 5%. This feature is often desirable in cavity occlusion devices since a body cavity, such as the LAA or an aneurysm, is relatively short and may vary from patient to patient. Many existing devices lengthen upon implantation due to radially compressive forces at the ostium or cavity wall which can affect proper positioning of the device.

Although the embodiment of an occlusion device 10 shown in FIGS. 6A-6B shows a planar or substantially planar proximal face 21, in some embodiments the low-profile proximal face 21 may have an arcuate, conical, and/or undulating contour. For example, FIG. 6E shows an embodiment of a frustum-shaped occlusion device 10 having an undulating proximal face 21. The lattice structure can be formed with a one-layer occlusive braid 16 having proximal ends coupled to a proximal region of a proximal hub 44 while the proximal ends of the structural braid 18 can be coupled to a distal region of the proximal hub 44. Accordingly, the proximal hub 44 is almost entirely encapsulated by a proximal region of the occlusive braid 16. As a result, the low-profile proximal face 21 is generally flat with a slight depression 25 along a longitudinal axis of the device 10. The slight depression 25 does not substantially disrupt the hemodynamics adjacent to the opening of the vascular structure (e.g., the left atrium, a blood vessel, etc.) nor have a significant effect on the profile of the proximal face 21. For example, in some embodiments, such bellows and/or undulations increase and/or decrease the profile of the proximal face 21 by less than 2 mm in the proximal direction. In other embodiments, such bellows and/or undulations increase and/or decrease the profile of the proximal face 21 by less than 1 mm in the proximal direction. As shown in FIG. 6E, the distal ends of the occlusive braid 16 and the distal ends of the structural braid 18 can be coupled to a proximal region of a distal hub 42. In some embodiments, the lattice structure can comprise a single layer including both occlusive and structural properties.

FIG. 6F shows a side view of another embodiment of an occlusion device 610 configured in accordance with the present technology. FIG. 6G is a cross-sectional side view of the occlusion device 610 shown in FIG. 6F. Referring to FIGS. 6F and 6G together, the occlusion device 610 is generally similar to the previously described occlusion device 10 (referenced herein with respect to FIGS. 6A-6E). The occlusion device 610 and/or occlusive braid 616 of the occlusion device 610, however, has a tapering distal region 624 with an atraumatic fastening element 656 at a distal end. Since the distal region 624 of the occlusion device 610 is defined by the distal region of the occlusive braid 616 (see FIG. 6G), the distal region 624 is highly flexible. Additionally, the atraumatic fastening element 656 not only lowers the risk of puncturing and/or damaging tissue at the vasculature structure, but also the fastening element 656 can be used as a capturing element should the device embolize and necessitate retrieval by the clinician (e.g., using a wire snare). Furthermore, the fastening element 656 may be radiopaque and help to better delineate the distal end of the device during placement. Although the fastening element 656 is shown as a spherical hollow structure in the illustrated embodiments, the fastening element 656 can be a solid structure and can have any suitable atraumatic shape.

The distal region 624 can have a first tier 650 extending distally from the contact portion, a second tier 652 extending distally from a distal section of the first tier 650, and a third tier 654 extending distally from the second tier 652 and terminating at the atraumatic fastening element 656. The first tier 650 and the second tier 652 can individually and/or cumulatively have a constantly decreasing diameter in a distal direction along a longitudinal axis L of the device 610. The slope of the first tier 650 can be steeper than the slope of the second tier 652. The third tier 654 can have a generally constant diameter along its length. In some embodiments, the occlusion device 610 can have less than three tiers (e.g., two tiers) or more than three tiers (e.g., four tiers, five tiers, etc.), and in some embodiments the occlusion device 610 can have any combination of the first, second, or third tiers. In yet other embodiments, the distal region can be a single tier (e.g., a cone) that extends distally from the contact portion with a linearly decreasing diameter.

FIG. 6H illustrates another embodiment of an occlusion device 500 that includes a proximal occlusion section 516, a distal occlusion section 518, and a core 520 between the proximal and distal occlusion sections 516 and 518 that is defined by the occlusive braid 501. The occlusion device 500 comprises a multi-layered occlusive braid 501 and two structural braids 503a, 503b within the occlusive braid 501 that individually correspond to the proximal occlusion section 516 and distal occlusion section 518, respectively. The proximal and distal occlusion sections 516 and 518 can have conical shapes with the peak of the proximal occlusion section 516 at a proximal hub 526 and the peak of the distal occlusion section 518 at a distal hub 532. The proximal and distal occlusion sections 516 and 518 can be a continuous layer of a single lattice structure and in some embodiments the proximal and distal occlusion sections 516 and 518 can be layers of the same or different lattice structures. For example, the proximal and distal occlusion sections 516 and 518 can have overlapping braided layers, interweaving layers, or fixed connections of one layer to another. FIG. 6I shows another embodiment of an occlusion device 550 that is similar to the occlusion device of FIG. 6H, but instead has single structural braid 503 enveloped by the multi-layered occlusive braid 501. As shown, the single structural braid 503 can have a core portion 552.

FIG. 6J illustrates an embodiment of the occlusion device 500 having a proximal section and a distal section positioned at a septal defect, such as an ASD (e.g., a PFO) or a VSD. As shown, the occlusion member 500 can further include tether 534 attached to the distal hub 532 such that the proximal and distal hubs 526 and 532 can be drawn together by the proximal retraction of the tether 534. A peripheral portion 520 of the first occlusion section 516 contacts one side of the septum (S) to cover one open end (O1) of the passage (P) and a peripheral portion of the second occlusion section 518 contacts the other side of the septum (S) to cover the opposite open end (O2) of the passage (P). For example, the tether 534 can be pulled such that it slides through the hubs 526 and 528 to draw the first and second occlusion sections 516, 518 against the opposing sides of the septum (S). This causes the lattice structures to press against the septum and cover the ends (O1) and (O2) of the passage (P).

FIG. 6K is a side view of yet another embodiment of an occlusion device 570 having a proximal occlusion section and a distal occlusion section positioned at a passage (P) through the septum (S) of the heart. In this particular embodiment, the lattice structure of the first occlusion section 516 is separate from the lattice structure of the second occlusion section 518. In several embodiments, the lattice structure can have at least one wire mesh, such as a wire braid, that has a disc-shape after implantation. The first occlusion section 516 can further include outer and inner hubs 526 and 528, respectively, connected to the ends of the lattice structure of the first occlusion member 516, and similarly the second occlusion member 518 can have outer and inner hubs 532 and 530 connected to the ends of the lattice structure of the second occlusion member 518. Each of the hubs 526, 528 and 530 can have a channel 533. The occlusion device 570 can further include a tether 534 that passes through the channels 533 of the hubs 526, 528 and 530, and a distal end of the tether 534 can be attached to the outer hub 532 of the second occlusion section 518.

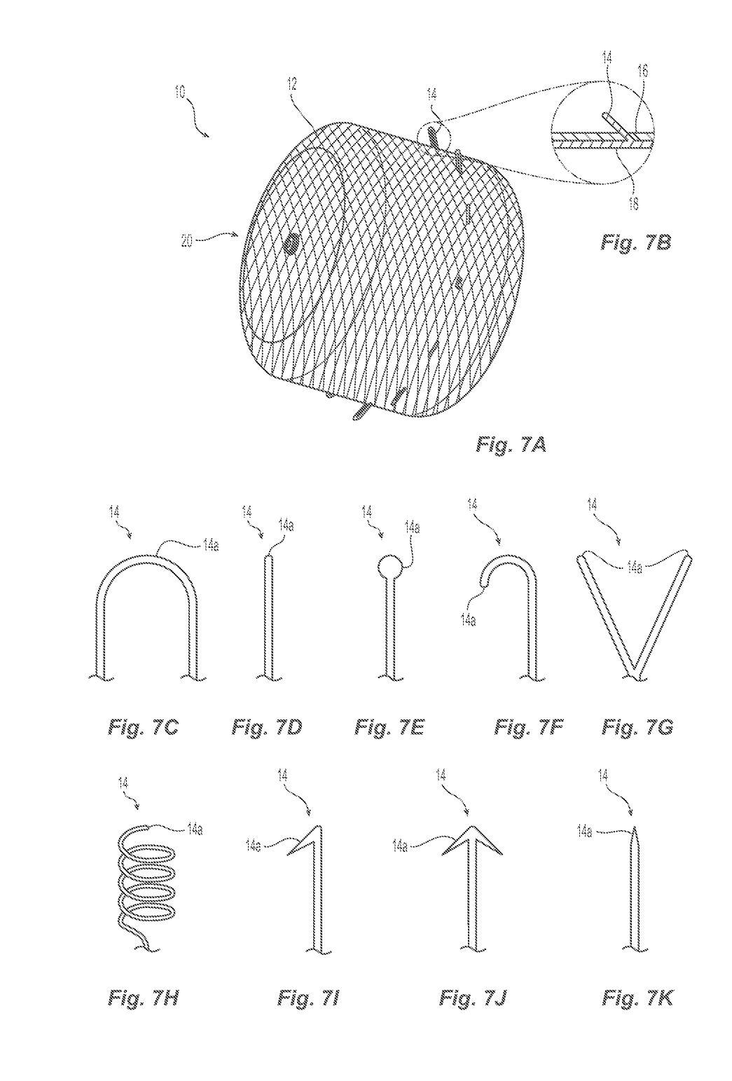

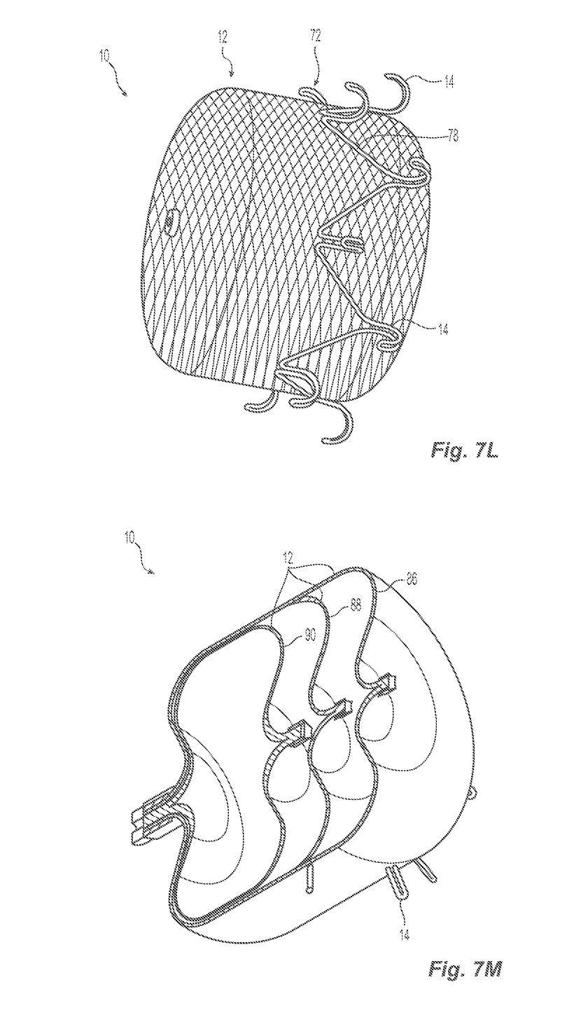

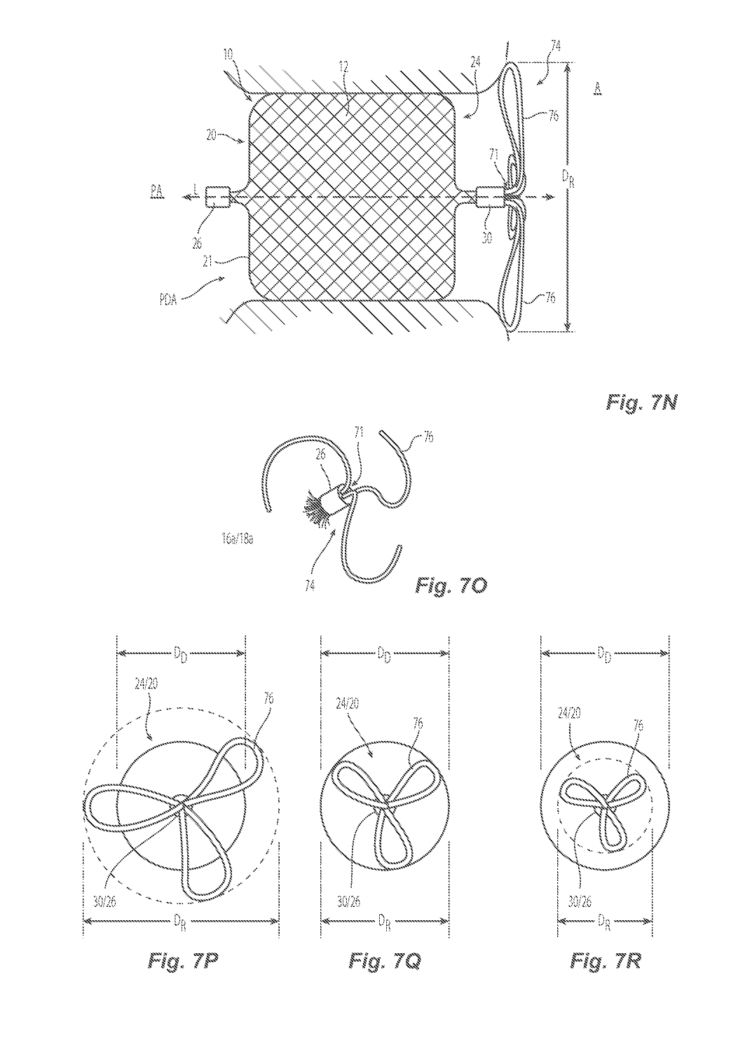

In some embodiments of the device, the occlusion device 10 may incorporate one or more atraumatic and/or non-tissue-penetrating retention members 14 to further secure the occlusion device 10 to at least a portion of the tissue at or near the vascular structure (e.g., the inner wall of the LAA, the right or left atrium walls, the right or left ventricle walls, etc.). FIGS. 7A-7B show one embodiment of an occlusion device 10 having retention members 14 arranged around the circumference of the device 10. As shown in the enlarged view of FIG. 7B, the retention members 14 may be contiguous or integrated with the structural braid 18 and pulled through the outer occlusive braid 16 to a point beyond the exterior of the device 10. Retention members 14 may be angled towards a proximal region 20 of the device 10 but are flexible enough to bend and/or conform in response to the local vascular structure anatomy.

Many existing devices fail to fully seal and/or fixate to the anatomy at a vascular structure, especially the portions of the vascular structure wall having protrusions (e.g., tissue, plaque, etc.) and thus fail to adequately secure the occlusion device at the vascular structure. To combat this issue, some existing devices include members with traumatic or tissue-penetrating shapes and/or ends coupled to the occlusion device. Such traumatic members may perforate the vascular structure walls causing pericardial effusion and even cardiac tamponade. To avoid these serious conditions, the retention members 14 of the present technology can have an atraumatic shape and are configured to capture and/or interface with the trabeculae without puncturing the trabeculae or the vascular structure walls. For example, FIGS. 7C-7G and show embodiments of retention members 14 having atraumatic shapes and/or ends 14a. The retention member 14 can be a u-shaped loop (FIG. 7C), a straight wire (FIG. 7D), a straight or bent wire with a spherical end 14a (FIG. 7E), a bent wire (FIG. 7F), a diverging wire have one or more ends 14a (FIG. 7G), and other suitable shapes and/or configurations.

In some embodiments, the occlusion device may additionally or alternatively include traumatic and/or tissue-penetrating retention members which can include at least one fixation member such as a tine, barb, hook (FIG. 71), pin (FIG. 7K), anchor (FIG. 7J) and others along at least a portion of the retention member 14 and/or at the end 14a of the retention member 14. In some embodiments, the length of the fixation members can be from about 0.025 mm to 0.5 mm. In other embodiments, the length of the fixation members can be about 0.5 mm to 2.0 mm. In some embodiments, the fixation members and/or retention members can include the use of additional expandable wires, struts, supports, clips, springs, glues, and adhesives. Some embodiments may include a vacuum.

FIG. 7L shows one embodiment of the occlusion device 10 having a separate retention structure 72 coupled to a lattice structure 12. The retention structure 72 can be made from a single wire, or may comprise more than one wire. The retention structure 72 can be secured to the lattice structure 12 and/or any layer of the lattice structure 12 by sewing, suturing, welding, mechanical coupling or any technique known in the art. The retention structure 72 includes non-penetrating retention members 14 attached by chevron-shaped struts 78 arranged circumferentially about the device 10. The chevron-shaped struts provide an array of retention members 14 within a circumferential band or zone of the cylindrical contact region 22 that can extend 2.0-20 mm along the length of the device 10. As shown in FIG. 7L, the retention members 14 can be atraumatic hooks. In other embodiments, the retention members 14 may include fixation members and/or any other suitable retention member shapes and/or configurations disclosed herein.

FIG. 7M shows another embodiment of the occlusion device 10 having a lattice structure 12 including three lattices—an anchoring lattice 86, an occlusive braid 88, and a structural braid 90. The anchoring lattice 86 can be a braid having at least two different filaments with different filament diameters such that portions of the larger filaments can be pulled away from the surface of the anchoring lattice 86 to form retention members 14. For example, in some embodiments, the anchoring lattice 86 may comprise two-thirds structural filaments having diameters between 0.001 in to 0.003 in, and one-third anchoring filaments having diameters between 0.003 in to 0.007 in.

Retention members may be located at any point along the surface of the occlusion device and could be in any arrangement (i.e., circumferentially and/or axially, etc.). The retention members and/or retention member associated structures can be constructed using metals, polymers, composites, and/or biologic materials. Polymer materials can include Dacron, polyester, polypropylene, nylon, Teflon, PTFE, ePTFE, TFE, PET, TPE, PLA silicone, polyurethane, polyethylene, ABS, polycarbonate, styrene, polyimide, PEBAX, Hytrel, poly vinyl chloride, HDPE, LDPE, PEEK, rubber, latex, or other suitable polymers. Metal materials can include, but are not limited to, nickel-titanium alloys (e.g. Nitinol), platinum, cobalt-chrome alloys, 35N LT, Elgiloy, stainless steel, tungsten or titanium. In some embodiments, the retention structure 72, retention members 14, occlusive braid 16 and/or structural braid 16 can comprise only metallic materials while the retention structure 72 and/or retention members 14 can be coupled to the occlusive 16 and/or structural braid 18 with a polymeric suture, fastener, or other suitable coupling means known in the art. Accordingly, the occlusion device can be substantially polymer free, that is, polymer free excluding the retention structure and/or retention member coupling means. In yet other embodiments, the occlusion device may not have retention members and is secured to the vascular structure by the radial and frictional forces of the structural braid 18.

Referring to FIGS. 7N-7O, in some embodiments the occlusion device 10 may include one or more terminal retention members 74 configured to stabilize and/or secure the occlusion device 10 at a target location at or within a vascular structure. As shown in FIG. 7N, the terminal retention member(s) 74 can be attached to the distal region 24 of the occlusion device 10 and have extensions 76 that project laterally with respect to the longitudinal dimension L of the device 10. In some embodiments, the occlusion device 10 can additionally or alternatively include one or more terminal retention member(s) 74 at the proximal region 20. The embodiment of the terminal retention member(s) 74 shown in FIG. 7N has a proximal end 71 attached to the distal outer hub 30, and the extensions 76 extend radially outwardly from the hub 30 to engage tissue positioned between the distal region 24 and the extensions 76. The extensions 76 can be radially expanding loops (FIG. 7N), spiraling elements (FIG. 7O), or any suitable shape and/or configuration. As shown in the schematic top views of FIGS. 7P-7R, the diameter of the terminal retention member 74 D_{R} can be generally larger than (FIG. 7P), equal to (FIG. 7Q), or smaller than (FIG. 7R) the diameter of the adjacent proximal or distal region D_{D}. In some embodiments the terminal retention member(s) 74 can engage tissue distal of the distal region 24 and/or proximal of the proximal region 20.

Referring back to FIG. 7N, the occlusion device 10 can be positioned at least partially within a patent ductus arteriosus (PDA). The terminal retention member 74 can protrude distally from a distal hub 30 and the extensions 76 radially extend to a terminal retention member diameter D_{R} that is greater than the inner diameter of the PDA. As a result, at least a portion of the extensions 76 engage the wall of the aorta (A) and prevent the occlusion device 10 from being pushed proximally through the PDA and into pulmonary circulation during systole.

The occlusion device may be constructed to elute or deliver one or more beneficial drug(s) and/or other bioactive substances into the blood or the surrounding tissue. For example, in some embodiments, the occlusion device may form or contain a reservoir to hold drug(s) and or other bioactive substances, and the occlusion device may include a valve for controlled release of such agents. The reservoir or drug containing portions may be dissolvable or contain dissolving components, including drug and/or structural components. The reservoir can release drugs by elution, diffusion, and/or mechanical actuation or electromechanical devices such as a pressurized gas chamber, a spring release, shape memory release, and/or temperature sensitive release systems.

In some embodiments, the reservoir may be refillable. Refilling drugs and/or actuating a gas or energy source may be by percutaneous hypodermic injection or by an intravascular catheter through a fitting or membrane. In some embodiments, the occlusion device may contain a collapsible reservoir configured to be delivered through an intravascular catheter. After delivery to a vascular structure, the collapsible reservoir can be expanded and fixed to an interior surface of the vascular structure.

The drugs and/or bioactive agents include an antiplatelet agent, including but not limited to aspirin, glycoprotein IIb/IIIa receptor inhibitors (including, abciximab, eptifibatide, tirofiban, lam ifiban, fradafiban, cromafiban, toxifiban, XV454, lefradafiban, klerval, lotrafiban, orbofiban, and xemilofiban), dipyridamole, apo-dipyridamole, persantine, prostacyclin, ticlopidine, clopidogrel, cromafiban, cilostazol, and nitric oxide. In any of the above embodiments, the device may include an anticoagulant such as heparin, low molecular weight heparin, hirudin, warfarin, bivalirudin, hirudin, argatroban, forskolin, ximelagatran, vapiprost, prostacyclin and prostacyclin analogues, dextran, synthetic antithrombin, Vasoflux, argatroban, efegatran, tick anticoagulant peptide, Ppack, HMG-CoA reductase inhibitors, thromboxane A2 receptor inhibitors, and others.

In some embodiments, the drugs and/or bioactive agents can be release directly into the left atrium. Directly releasing drugs into the heart circulation is advantageous because it requires a lower dose, increases effectiveness, lowers side effects, improves the safety profile, localizes delivery, bypasses the digestive system, substitutes for intravenous or intra-arterial injection, substitutes for oral ingestion, and others. In some embodiments, drug release following implant would be limited to an initial time period of less than five years. In other embodiments, drug release following implant would be limited to an initial time period of less than 1 year. In yet other embodiments, drug release following implant would be limited to an initial time period of less than 3 to 6 months, or in some embodiments, less than 45 days.

In some embodiments, one or more eluting filament(s) may be interwoven into the lattice structure 12 to provide for the delivery of drugs, bioactive agents or materials with a mild inflammatory response as disclosed herein. The interwoven filaments may be woven into the lattice structure after heat treating (as discussed below) to avoid damage to the interwoven filaments by the heat treatment process. In some embodiments, the occlusion device may be coated with various polymers to enhance its performance, fixation and/or biocompatibility. In other embodiments, the device may incorporate cells and/or other biologic material to promote sealing, reduction of leakage and/or healing.

### 2. Delivery Systems and Methods

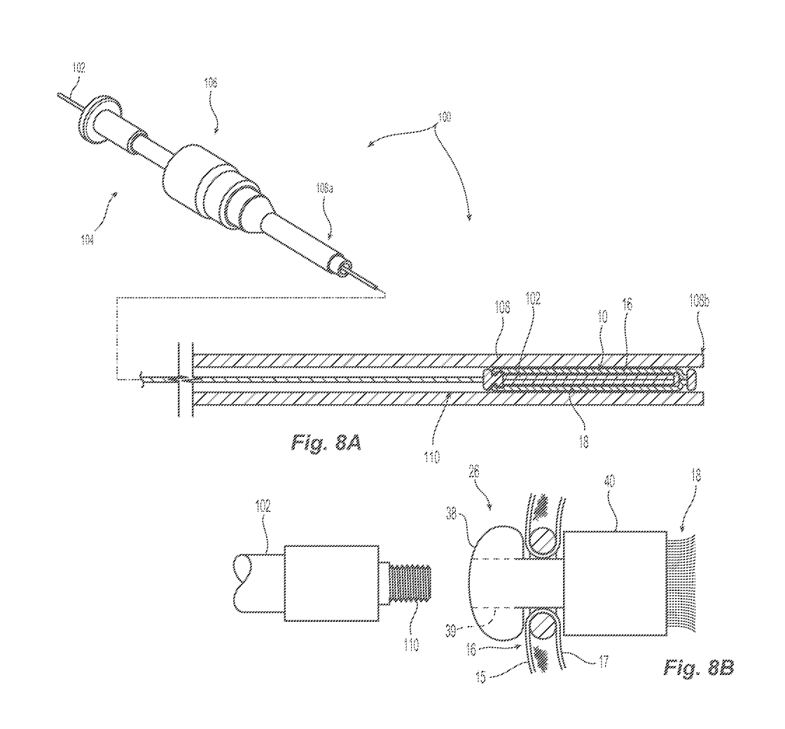

FIGS. 8A-12B illustrate embodiments of a delivery system 100 and methods for deploying the occlusion device 10. FIG. 8A is a cross-sectional side view of one embodiment of the delivery system 100 showing the occlusion device 10 in a collapsed, low-profile configuration for percutaneous delivery. The delivery system 100 may include a guidewire (not shown), a detachment system 110, and a single or multi-lumen delivery catheter 104 having a proximal hub 106 and a sheath 108. The sheath 108 has a distal zone 108b, a proximal zone 108a, and a lumen therethrough. For example, the lumen of the sheath 108 can have a diameter between 6F and 30F, and in some embodiments, between 8F and 12F.

As shown in FIG. 8B, the detachment system 110 can include a torque cable 102 coupled to screw threads 109 at a distal end of the torque cable 102. The screw threads 109 can match the internal threads of a hole 39 in the locking member 38 of the proximal hub 26 of the device 10 such that unscrewing the screw threads 109 releases the proximal hub 26 from the detachment system 110. In some embodiments, the detachment system may comprise a tether coupled to an electronic system that upon application of an electrical current to the tether severs the tether and releases the device.

Access to the desired vascular structure can be accomplished through the patient's vasculature in a percutaneous manner. By percutaneous it is meant that a location of the vasculature remote from the heart is accessed through the skin, typically using a surgical cut down procedure or a minimally invasive procedure, such as using needle access through, for example, the Seldinger technique. The ability to percutaneously access the remote vasculature is well-known and described in the patent and medical literature. Once percutaneous access is achieved (for example, through the femoral or iliac veins), the interventional tools and supporting catheter(s) may be advanced to the target site intravascularly and positioned within or near the vascular structure in a variety of manners, as described herein.

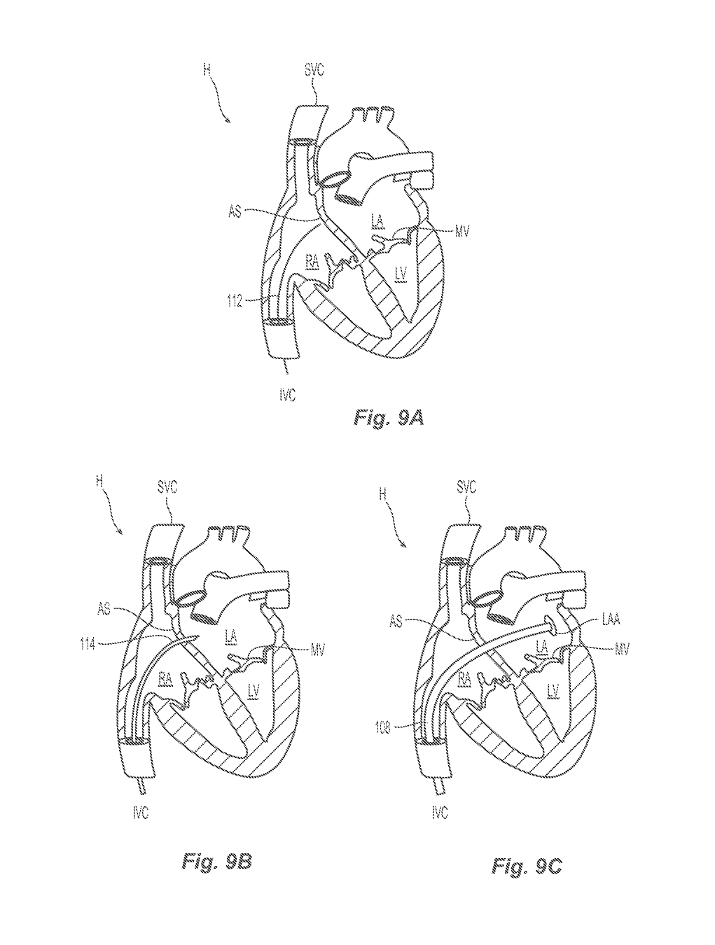

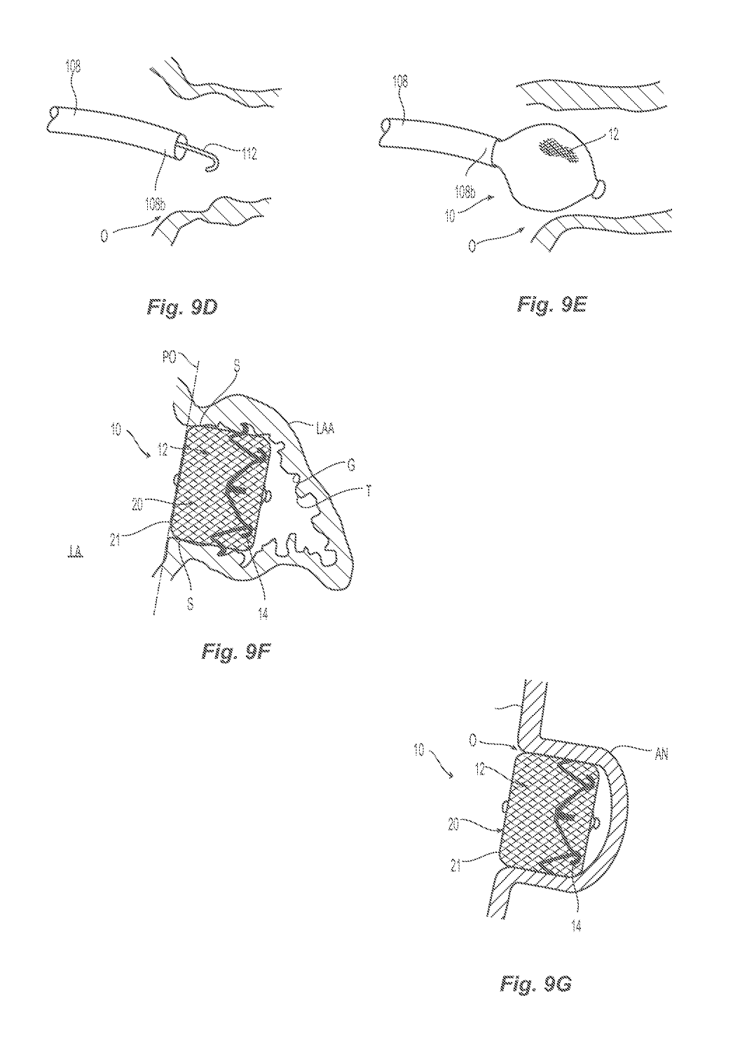

FIGS. 9A-9B illustrate one example for positioning an occlusion device in the right atrium (RA) and/or left atrium (LA) of the heart using an antegrade approach. As shown in FIG. 9A, a guidewire 112 may be advanced intravascularly using any number of techniques, e.g., through the inferior vena cava (IVC) or superior vena cava (SVC) (not shown) into the right atrium (RA). If access to the left atrium (LA) is desired (e.g., ASD closures), the guidewire 112 may be exchanged for a needle 114. As shown in FIG. 9B, the needle 114 punctures the atrial septum (AS) of the heart to gain access to the left atrium (LA). The needle 114 is then removed proximally. Alternatively, the device 10 may be passed through a PFO or other existing ASD to the left atrium (LA).

The delivery sheath 108 containing the collapsed occlusion device 10 and detachment system 110 can be advanced together with the guidewire 112 (i.e., using an over the wire or a rapid exchange catheter system) until the distal zone 108b of the catheter is positioned at or near a target location at or within a vascular structure opening, such as just distal to the LAA ostium or PFO. The guidewire 112 and catheter 108 can be advanced through the vasculature using known imaging systems and techniques such as fluoroscopy, x-ray, MRI, ultrasound or others. Radiopaque markers (not shown) can be incorporated into the guidewire 112, needle 114, detachment system 110, catheter 104, sheath 108, and/or the occlusion device 10 itself to provide additional visibility under imaging guidance. Such marker materials can be made from tungsten, tantalum, platinum, palladium, gold, iridium, or other suitable materials.

After the distal zone 108b of the sheath 108 is at or proximal to the vascular structure opening, the guidewire 112 is removed proximally through the lumen of the delivery catheter 104. Next, the sheath 108 is retracted proximally and an exposed portion of the occlusion device 10 expands (FIG. 9E) such that a portion of the occlusion device 10 contacts tissue along at least a portion of an entrance region of the targeted vascular structure. For example, in LAA applications, the occlusion device 10 contacts the ostium O and/or the LAA wall along at least a portion of a smooth entrance region S of the LAA, as shown in FIG. 9F. In some embodiments, the occlusion device 10 may be actively expanded using conventional techniques known in the art, such as pull-wires attached to a distal end of the device and/or a balloon assembly.

During deployment, the detachment system 110 engages the cap 38 to facilitate deployment of the occlusion device 10. After deployment is completed, the detachment system 110 can disengage from the cap 38 (see FIG. 8B) by unscrewing (i.e., rotating a proximal end of the torque cable 102). In other embodiments, other release mechanisms and/or couplings may be used, including hydraulic, electrothermal, electroresistive, electrolytic, electrochemical, electromechanical and mechanical release mechanisms.

FIGS. 9F-9H show the occlusion device 10 implanted in the various anatomical locations with retention members 14 interfacing with the vascular structure such that the proximal face 21 of the proximal portion 20 of the occlusion device 10 is substantially within or just proximal to the plane of the vascular structure opening O. The fully expanded circumference of the lattice structure 12 may be selected to exceed the circumference of the vascular structure opening in order to increase radial force after placement for promoting fixation and sealing. In some embodiments, the maximum expansion of the lattice structure 12 is controlled to expand to the diameter of the vascular structure and/or vascular structure opening.

FIG. 9F shows the LAA often has a “chicken wing” morphology that makes it difficult to properly position, secure and seal existing transcatheter occlusion devices. Just distal to the LAA ostium O is a short LAA entrance region S having relatively smooth inner walls. If the proximal end of an occlusion device is positioned too distal to the ostium, the device is likely to turn out of plane of the ostium PO and/or fall deeper into the LAA. Such unwanted repositioning can create a gap between the plane of the ostium PO and the proximal end of the device and/or the proximal end of the device may sit at an angle with respect to the plane of the ostium PO. Such gaps and/or corners/bends/crooks in the device can be potential locations of thrombus formation that defeat the purpose of the occlusion device. FIG. 9G is a side perspective view of the occlusion device in a deployed state positioned at an aneurysm (AN) such that the proximal face 21 of the proximal portion 20 of the occlusion device 10 is substantially within or just proximal to the plane of the aneurysm opening O.

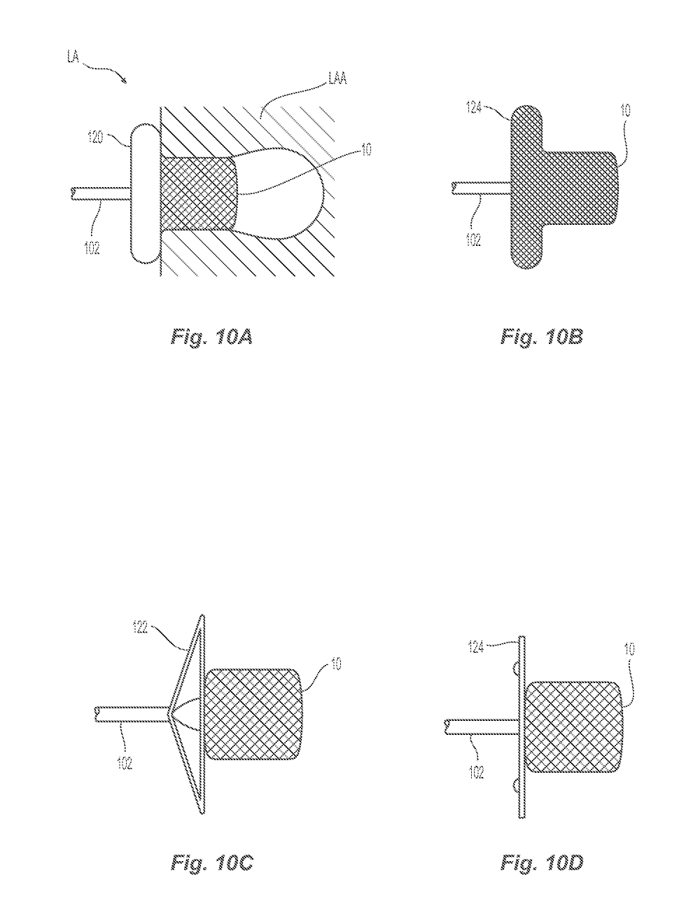

FIGS. 10A-10D show several embodiments in which the delivery system may include one or more positioning members to facilitate positioning a proximal region of the occlusion device 10 in substantial alignment with the plane of the vascular structure opening O. For example, as shown in FIG. 10A, the distal region of the delivery system may include a balloon 120 proximal to the occlusion device 10. The balloon 120 can be configured to expand to a diameter greater than the diameter of the vascular structure opening such that the balloon 120 abuts the tissue surrounding the vascular structure opening. In some embodiments, the occlusion device is expanded or partially expanded and then the balloon is expanded and positioned against the ostium.

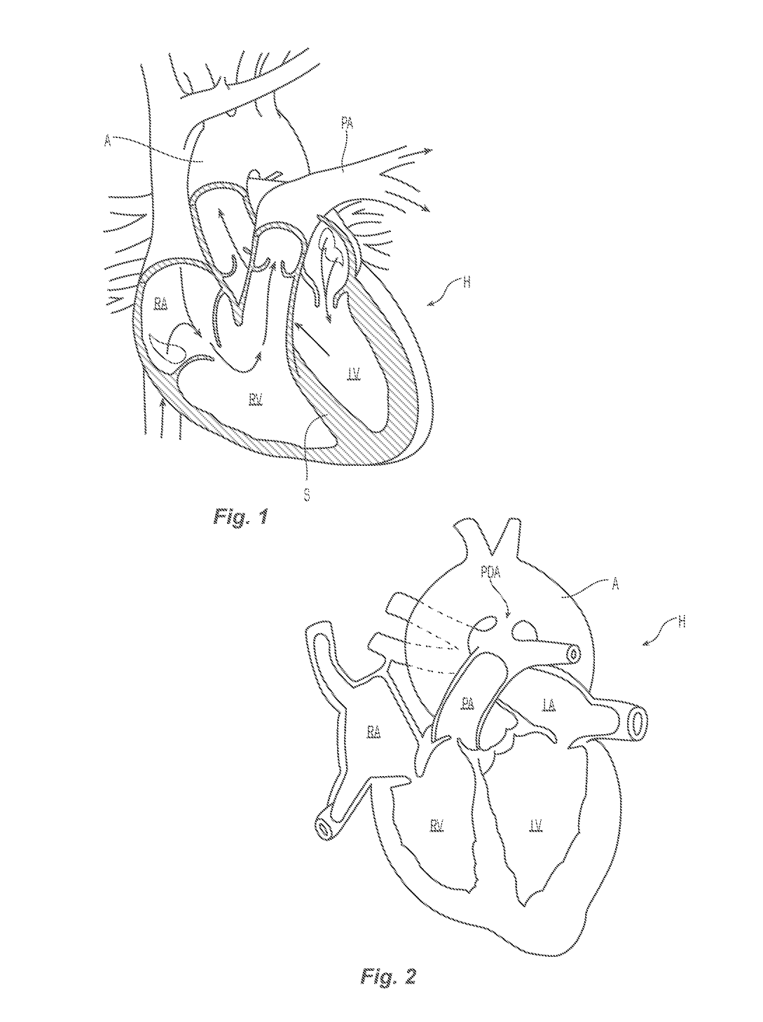

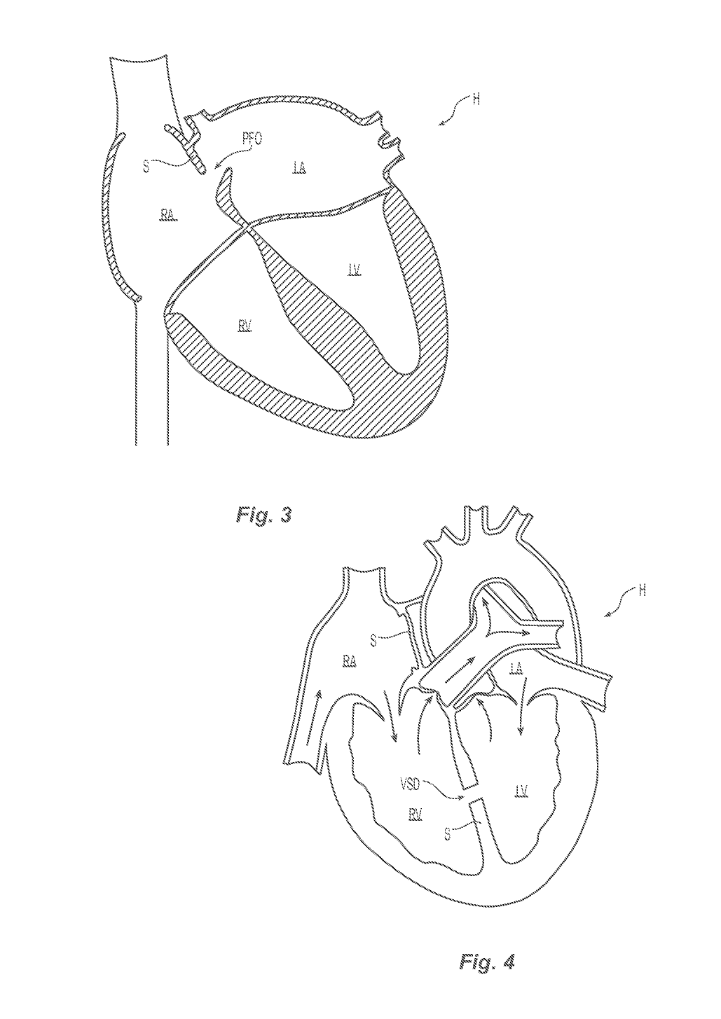

The balloon 120 can be non-compliant or compliant and can have an oblate spheroid, spheroid, spheroid with a flattened side proximate the ostium, or other suitable shapes. In one embodiment, the occlusion device 10 and balloon 120 are inserted intravascularly to a position at or near the target vascular structure and initially positioned inside the vascular structure using imaging modalities including TEE, fluoroscopy, CT, and others. The balloon may be filled with a contrast medium to aid in visualization and/or radiopaque markers may be placed on the balloon, catheter or occlusion device to aid in visualization before, during and after placement. The balloon is deflated prior to removal from the vasculature. In some embodiments, other positioning structures may be used in addition to or in place of the balloon, including an expandable braided mesh (FIG. 10B), an expandable Malecot structure (FIG. 10C), a mechanical positioner (FIG. 1 OD), or other suitable positioning structures.

### 3. Lattice Structure and Formation

In any of the embodiments described herein, the lattice structure and/or layers comprising the lattice structure can be a latticework, mesh, and/or braid of wires, filaments, threads, sutures, fibers or the like, that have been configured to form a fabric or structure having openings (e.g., a porous fabric or structure). The mesh can be constructed using metals, polymers, composites, and/or biologic materials. Polymer materials can include Dacron, polyester, polypropylene, nylon, Teflon, PTFE, ePTFE, TFE, PET, TPE, PLA silicone, polyurethane, polyethylene, ABS, polycarbonate, styrene, polyimide, PEBAX, Hytrel, poly vinyl chloride, HDPE, LDPE, PEEK, rubber, latex, or other suitable polymers. Other materials known in the art of elastic implants can also be used. Metal materials can include, but are not limited to, nickel-titanium alloys (e.g. Nitinol), platinum, cobalt-chrome alloys, 35N LT, Elgiloy, stainless steel, tungsten or titanium. In certain embodiments, metal filaments may be highly polished or surface treated to further improve their hemocompatibility. In some embodiments, it is desirable that the mesh be constructed solely from metallic materials without the inclusion of any polymer materials, i.e., polymer free. In these embodiments and others, it is desirable that the entirety of the occlusion device be made of metallic materials free of any polymer materials. It is believed that the exclusion of polymer materials in some embodiments may decrease the likelihood of thrombus formation on device surfaces, and it is further believed that the exclusion of polymers and the sole use of metallic components can provide an occlusion device with a thinner profile that can be delivered with a smaller catheter as compared to devices having polymeric components.

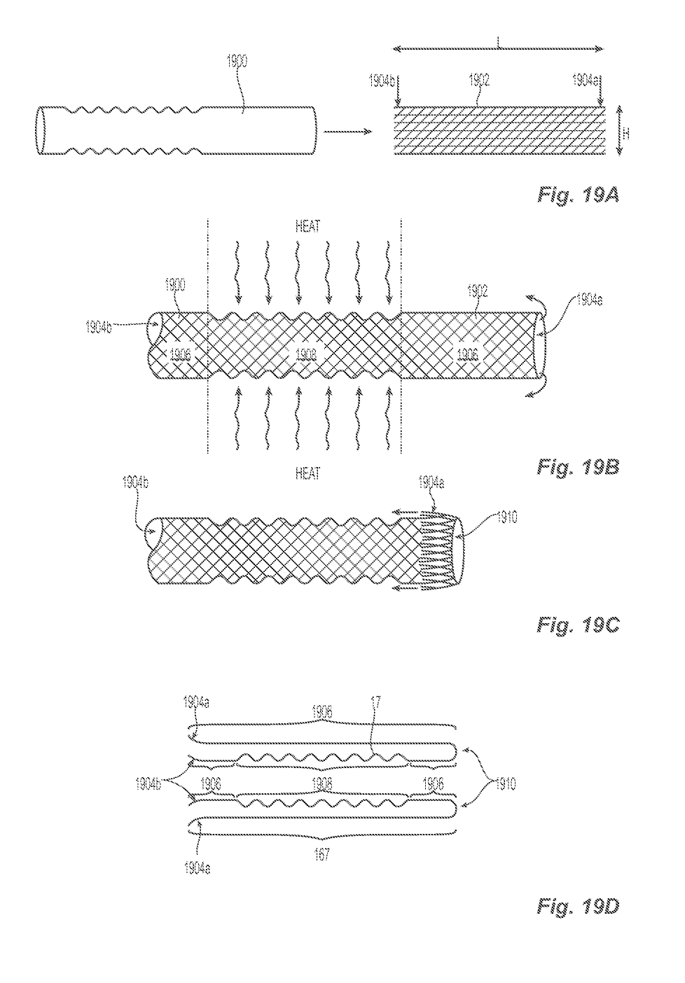

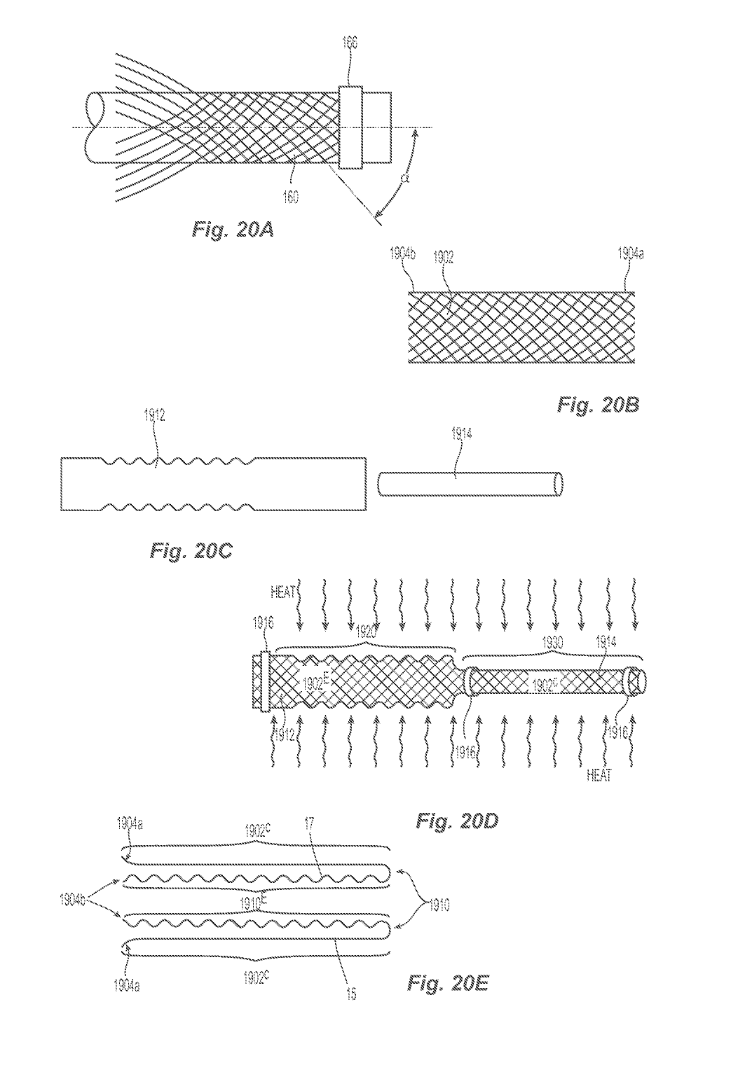

FIG. 11A shows the lattice structure and/or lattices comprising the lattice structure being formed over a mandrel 160 (e.g., a fixture, a mold, etc.) as is known in the art of tubular braid manufacturing. The braid angle alpha a can be controlled by various means known in the art of filament braiding, as described in great detail below. The tubular braided mesh can then be further shaped using a heat setting process. As is known in the art of heat setting a braiding filament such as Nitinol wires, a mandrel 160 and one or more collars 166 positioned on the mandrel 160 can be used to hold the braided tubular structure in its desired configuration while subjected to an appropriate heat treatment such that the resilient filaments of the braided tubular member assume or are otherwise shape-set to the outer contour of the mandrel 160. The filamentary elements of a mesh device or component can be held by a mandrel 160 configured to hold the device or component in a desired shape and, in the case of Nitinol wires, heated to about 475-525° C. for about 5-30 minutes to shape-set the structure. Other heating processes are possible and can depend on the properties of the material selected for braiding. In some embodiments, the heat setting process can be applied to select portions of the braid, and in some embodiments the heat setting process can be applied while the braid is held in an expanded and/or contracted state (described in more detail below with respect to FIGS. 19A-20E).

For braided portions, components, or elements, the braiding process can be carried out by automated machine fabrication or can also be performed by hand. For some embodiments, the braiding process can be carried out by the braiding apparatus and process described in U.S. Pat. No. 8,261,648, filed Oct. 17, 2011 and entitled “Braiding Mechanism and Methods of Use” by Marchand et al., which is herein incorporated by reference in its entirety. In some embodiments, a braiding mechanism may be utilized that comprises a disc defining a plane and a circumferential edge, a mandrel extending from a center of the disc and generally perpendicular to the plane of the disc, and a plurality of actuators positioned circumferentially around the edge of the disc. A plurality of filaments are loaded on the mandrel such that each filament extends radially toward the circumferential edge of the disc and each filament contacts the disc at a point of engagement on the circumferential edge, which is spaced apart a discrete distance from adjacent points of engagement. The point at which each filament engages the circumferential edge of the disc is separated by a distance “d” from the points at which each immediately adjacent filament engages the circumferential edge of the disc. The disc and a plurality of catch mechanisms are configured to move relative to one another to rotate a first subset of filaments relative to a second subset of filaments to interweave the filaments. The first subset of the plurality of filaments is engaged by the actuators, and the plurality of actuators is operated to move the engaged filaments in a generally radial direction to a position beyond the circumferential edge of the disc. The disc is then rotated a first direction by a circumferential distance, thereby rotating a second subset of filaments a discrete distance and crossing the filaments of the first subset over the filaments of the second subset. The actuators are operated again to move the first subset of filaments to a radial position on the circumferential edge of the disc, wherein each filament in the first subset is released to engage the circumferential edge of the disc at a circumferential distance from its previous point of engagement.

In some embodiments, the lattice structure and/or layers of the lattice structure may be formed using conventional machining, laser cutting, electrical discharge machining (ECM) or photochemical machining (PCM). In some embodiments, the lattice structure and/or layers of the lattice structure may be formed from metallic tubes and/or sheet material. Some PCM processes for making similar structures are described in U.S. Pat. No. 5,907,893, filed Jan. 31, 1997 entitled “Methods for the Manufacture of Radially Expansible Stents” by Zadno-Azizi et al., and in U.S. Pat. No. 7,455,753, filed Oct. 10, 2006 entitled “Thin Film Stent” by Roth, which are both herein incorporated in their entirety by reference.

The terms “formed,” “preformed,” and “fabricated” may include the use of molds or tools that are designed to impart a shape, geometry, bend, curve, slit, serration, scallop, void, hole in the elastic, superelastic, or shape memory material or materials used in the components of the occlusion device, including the mesh. These molds or tools may impart such features at prescribed temperatures or heat treatments.

The filaments of the braids can be arranged in a generally axially elongated configuration when the occlusion device 10 is within the delivery catheter. In the expanded or deployed configuration, certain embodiments of the filaments have a “low” filament braid angle “a” from about 5 to 45 degrees with respect to the longitudinal axis of the device (see FIG. 11A) such that the filaments are angled toward the longitudinal dimension of the occlusion device 10. In some embodiments, the filaments can have a “high” braid angle α between about 45 to 85 degrees with respect to the longitudinal axis of the occlusion device. The braids for the mesh components can have a generally constant braid angle α over the length of a component or can be varied to provide different zones of pore size and radial stiffness. The expanded braided mesh can conform to or otherwise contact the vessels without folds along the longitudinal axis. The cross-sectional dimension of the lattice structure in the expanded state can be from 3 mm to 60 mm, or from 10 mm to 40 mm in some embodiments. The diameters of the lattice structure within the delivery catheter can be about 1 mm to 15 mm, or 5 mm to 10 mm in more specific applications.

In some embodiments, braid filaments of varying diameters may be combined in the same layer of the lattice or portions of the lattice to impart different characteristics including, e.g., stiffness, elasticity, structure, radial force, pore size, embolic filtering ability, and/or other features. For example, in the embodiment shown in FIG. 11B, the braided mesh has a first mesh filament diameter 164 and a second mesh filament diameter 165 smaller than the first mesh filament diameter 164. In some embodiments, the diameter of the structural 18 and/or occlusive 16 braid filaments can be less than about 0.5 mm. In other embodiments, the filament diameter may range from about 0.01 mm to about 0.40 mm. In some embodiments, the thickness of the structural braid 18 filaments would be less that about 0.5 mm. In some embodiments, the structural braid 18 may be fabricated from wires with diameters ranging from about 0.015 mm to about 0.25 mm. In some embodiments, the thickness of the occlusive braid 16 filaments would be less that about 0.25 mm. In some embodiments, the occlusive braid 16 may be fabricated from wires with diameters ranging from about 0.01 mm to about 0.20 mm.

As used herein, “pore size” refers to the diameter of the largest circle 162 that fits within an individual cell of a braid (see FIG. 11C). The average and/or maximum pore size of the structural braid 18 can be greater than 0.20 mm, and generally more than 0.25 mm. The structural braid 18 or portions of the structural braid 18 are configured to provide stability and exert radial forces that secure and shape other layers and/or braids of the lattice structure 12 to surrounding tissue structures. The radial force exerted by the structural braid 18 is generally sufficient to inhibit movement, dislodgement and potential embolization of the occlusion device 10. For the occlusive braid 16, average and/or maximum pore sizes in the range of about 0.025 mm to 2.0 mm may be utilized. In some embodiments, the occlusive braid 16 average and/or maximum pore sizes may be in the range of 0.025 mm to 0.300 mm, outside the range of existing devices. Likewise, the radial stiffness of the structural braid 18 can be 10-100 times greater than the radial stiffness of the occlusive braid 16. In some embodiments, the radial stiffness of the structural braid 18 is 10-50 times greater than the radial stiffness of the occlusive braid 16.

Different layers of the lattice structure 12 may have different filament counts. In some embodiments, the braided filament count for the occlusive braid 16 is greater than 290 filaments per inch. In one embodiment, the braided filament count for the occlusive braid 16 is between about 360 to about 780 filaments, or in further embodiments between about 144 to about 290 filaments. In one embodiment, the braided filament count for the structural braid 18 is between about 72 and about 144 filaments, or in other embodiments between about 72 and about 162 filaments. In some embodiments, the device 10 may include polymer filaments or fabric within the lattice layers 16, 18 or between layers of braids.

For some embodiments, three factors are often desirable for a woven or braided wire occlusion device that can achieve a desired clinical outcome in the endovascular treatment of abnormal vascular structure disorders such as LAA, PFO, VSD, and others. For effective use in some applications, it may be desirable for the occlusion device to have sufficient radial stiffness for stability, limited pore size for rapid promotion of hemostasis leading to occlusion, and a collapsed profile which is small enough to allow insertion through an inner lumen of a vascular catheter. A device with a radial stiffness below a certain threshold may be unstable and may be at higher risk of movement or embolization in some cases. Larger pores between filament intersections in a braided or woven structure may not generate thrombi and cause occlusion in an acute setting and thus may not give a treating physician or health professional such clinical feedback that the flow disruption will lead to a complete and lasting occlusion of the vascular structure being treated. Delivery of a device for treatment of a patient's vasculature through a standard vascular catheter may be highly desirable to allow access through the vasculature in the manner that a treating physician is accustomed.

The “average maximum pore size” in a portion of a device that spans an opening of the vascular structure, such as the LAA ostium, is desirable for some useful embodiments of a braided wire device for treatment and may be expressed as a function of the total number of all filaments, filament diameter and the device diameter. As used in the equation below and accompanying discussion, “average maximum pore size” refers to an average pore size of the “M” largest pore sizes (LPS) in the portion of the device that spans an opening in the vascular structure, where M is a positive integer that varies based on the device (see FIG. 11D). For example, in some devices, it may be appropriate to select an M of 10. In this case, the ten largest pore sizes in the portion of the device that spans an opening in the vascular structure would be averaged to determine the average maximum pore size in that portion of the device. The difference between filament sizes, where two or more filament diameters or transverse dimensions are used, may be ignored in some cases for devices where the filament size(s) are very small compared to the device dimensions. For a two-filament device, the smallest filament diameter may be used for the calculation. Thus, the average maximum pore size for such embodiments may be expressed as follows:

Pₘₐₓ=(1.7/NT)*(pD−(NTdw/2));

where Pₘₐₓ is the average maximum pore size;

D is the device diameter (transverse dimension);

NT is the total number of all filaments; and

dw is the diameter of the filaments (smallest) in inches.

Using this expression, the average maximum pore size, Pₘₐₓ, of the of the device may be less than about 0.016 inches or about 400 microns for some embodiments. In some embodiments the average maximum pore size of the device may be less than about 0.012 inches or about 0.300 mm. In some embodiments, the average maximum pore size of the device can be between 0.1 mm to 0.3 mm. In other embodiments, the average maximum pore size of the device can be between 0.075 mm to 0.250 mm.

The collapsed profile of a two-filament (profile having two different filament diameters) braided filament device may be expressed as the function:

P_{c}=1.48((Nₗdₗ²+Nₛdₛ²))^{1/2};

where P_{c} is the collapsed profile of the device;

Nₗ is the number of large filaments;

Nₛ is the number of small filaments;

dₗ is the diameter of the large filaments in inches; and

dₛ is the diameter of the small filaments in inches.

Using this expression, the collapsed profile P_{c} may be less than about 4.0 mm for some embodiments of particular clinical value. In some embodiments of particular clinical value, the device may be constructed so as to have both factors (Pₘₐₓ and P_{c}) above within the ranges discussed above; Pₘₐₓ less than about 300 microns and P_{c} less than about 4.0 mm, simultaneously. In some such embodiments, the device may be made to include about 200 filaments to about 800 filaments. In some cases, the filaments may have an outer transverse dimension or diameter of about 0.0008 inches to about 0.012 inches.

In some embodiments, a combination of small and large filament sizes may be utilized to make a device with a desired radial compliance and yet have a collapsed profile which is configured to fit through an inner lumen of commonly used vascular catheters. A device fabricated with even a small number of relatively large filaments can provide reduced radial compliance (or increased stiffness) compared to a device made with all small filaments. Even a relatively small number of larger filaments may provide a substantial increase in bending stiffness due to change in the moment of Inertia (I) that results from an increase in diameter without increasing the total cross sectional area of the filaments. The moment of inertia (I) of a round wire or filament may be defined by the equation:

I=πd⁴;

where d is the diameter of the wire or filament.

Since the moment of inertia is a function of filament diameter to the fourth power, a small change in the diameter greatly increases the moment of inertia. Thus, a small change in filament size can have substantial impact on the deflection at a given load and thus the compliance of the device.

Thus, the stiffness can be increased by a significant amount without a large increase in the cross sectional area of a collapsed profile of the device. This may be particularly important as device embodiments are made larger to treat larger vascular structures. As such, some embodiments of devices for treatment of a patient's vasculature may be formed using a combination of filaments with a number of different diameters such as 2, 3, 4, 5 or more different diameters or transverse dimensions. In device embodiments where filaments with two different diameters are used, some larger filament embodiments may have a transverse dimension of about 0.004 inches to about 0.012 inches and some small filament embodiments may have a transverse dimension or diameter of about 0.0008 inches and about 0.003 inches. The ratio of the number of large filaments to the number of small filaments may be between about 4 to 16 and may also be between about 6 to 10. In some embodiments, the difference in diameter or transverse dimension between the larger and smaller filaments may be less than about 0.008 inches. In some embodiments, less than about 0.005 inches, and in other embodiments, less than about 0.003 inches.

For some embodiments, it may be desirable to use filaments having two or more different diameters or transverse dimensions to form a permeable shell in order to produce a desired configuration as discussed in more detail below. The radial stiffness of a two-filament (two different diameters) woven device may be expressed as a function of the number of filaments and their diameters, as follows:

S_{radial}=(1.2×10⁶ lbf/D⁴)*(Nₗdₗ⁴+Nₛdₛ⁴);

where S_{radial} is the radial stiffness in pounds force (lbf);

D is the device diameter (transverse dimension);

Nₗ is the number of large filaments;

Nₛ is the number of small filaments;

dₗ is the diameter of the large filaments in inches; and

dₛ is the diameter of the small filaments in inches.

Using this expression, the radial stiffness, S_{radial} may be between about 0.014 and 0.284 lbf force for some embodiments of particular clinical value.

### 4. Occlusion Device Shapes and Layering

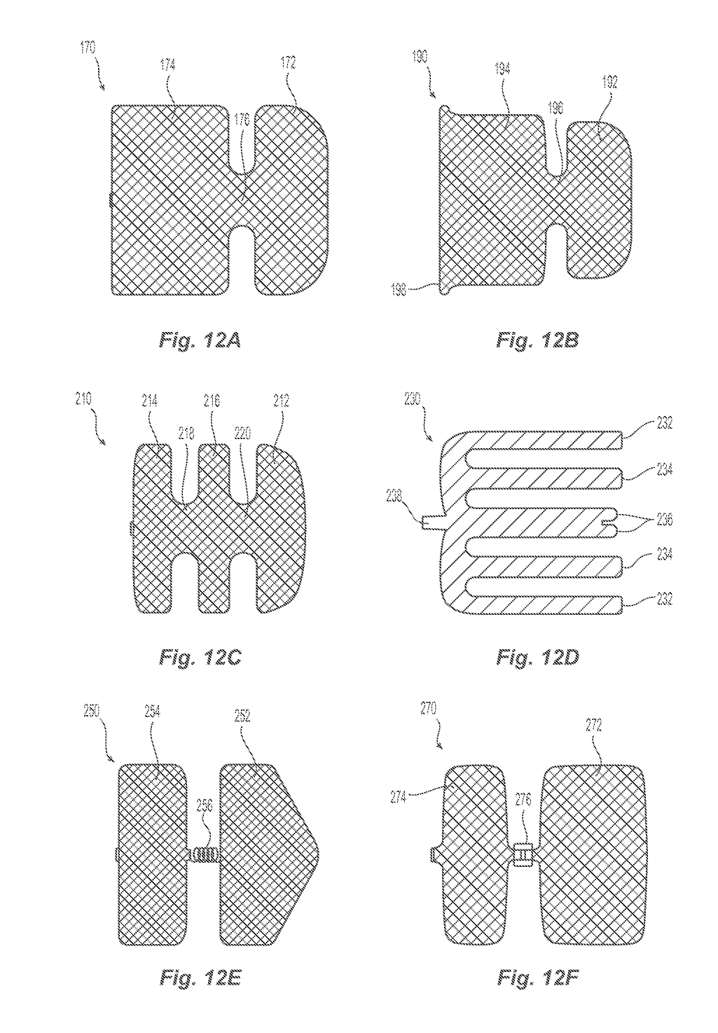

Several configurations of occlusion devices and/or lattice structure shapes are described in the following embodiments. As can be appreciated, the described features or combination of features for a particular embodiment can be applied to another embodiment. Furthermore, for clarity, features that are common to earlier-described embodiments are not again described in detail with reference to FIGS. 12A-23C as reference can be made to those features in earlier descriptions. For example, although only the outermost layer is shown in the lattice structures illustrated in FIGS. 12A-23C, any of the lattice structure sections described below can comprise one or more braided layers along its entire length or a portion of its length.

FIG. 12A illustrates an embodiment of a lattice structure 170 having a proximal section 174 and a distal section 172 connected to the proximal section 174 by a connecting section 176. The proximal section 174 fixates and seals the device 170 to the ostium and/or LAA while the distal section 172 extends into the LAA cavity and further fixates the device. The connecting section 176 facilitates flexing of the lattice structure 170 along its central longitudinal axis so as to adjust to one or more lobes of the LAA. In some embodiments the proximal and/or distal sections 174 and 172 can have an oval shape or other shapes to conform to the geometry of the LAA ostium and appendage body.

In some embodiments, the radial stiffness of the distal section may be substantially less than the radial stiffness of the proximal section. Accordingly, the distal section may be much more compliant than the proximal section to conform to anatomical variations often found in the LAA. The malleability of the distal section improves surface area contact with the LAA walls and/or trabeculae and resists movement. In some embodiments, the radial stiffness of the proximal section may be between about 1.5 times to 5 times the radial stiffness of the distal section.

Referring to FIG. 12B, the lattice structure can have a flange 198 at a proximal edge of the proximal section 194. When deployed, the flange 198 is positioned in contact with the left atrium wall at or slightly proximal to the ostium of the LAA. The flange 198 is expected to align the proximal face of the device 10 with the plane of the LAA ostium. This may assist in preventing the device 10 from turning out of the plane of the LAA ostium.

In other embodiments, the lattice structure can have more than two lattice sections. For example, FIG. 12C shows one embodiment of an occlusion device having a proximal section 214, a middle section 216, and a distal section 212. The proximal section 214 connects to the middle section 216 through a first connector 218, and the middle section connects to the distal section through a second connector 220. FIG. 12D shows another embodiment of a lattice structure 230 having a plurality of annular lattice sections including, for example, an outer ring 232, an intermediate ring 234, and an inner ring 236.

In some embodiments, the sections of the lattice structure may be coupled by a connector. For example, as shown in FIG. 12E, a lattice structure 250 can have a proximal section 254 and a distal section 252 coupled by a spring 256. In other embodiments, the connector can be a mechanical coupling 276, as shown in FIG. 12F.

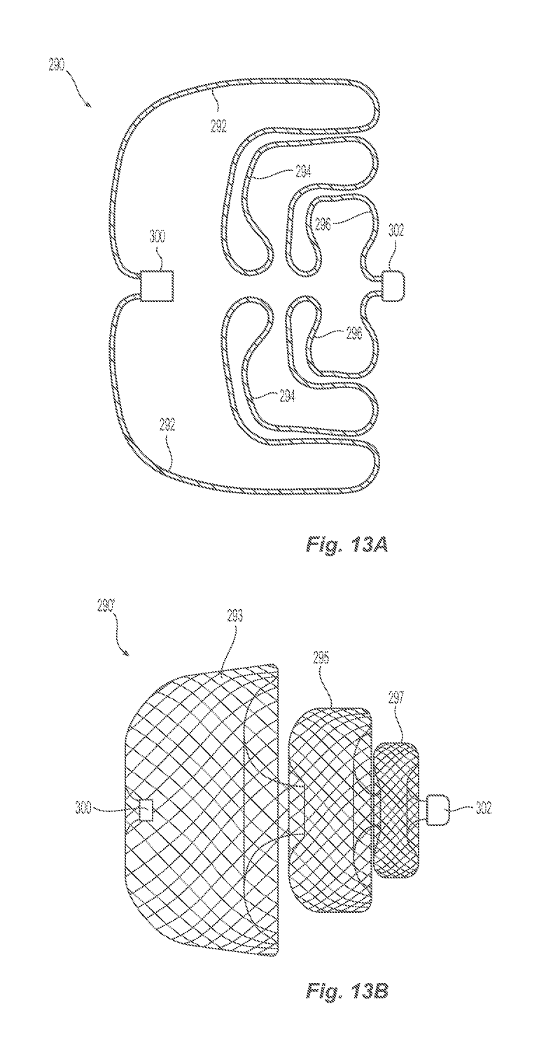

Referring to FIGS. 13A-13B, in some embodiments the lattice structure may have nested sections. As shown in the cross-sectional side view of FIG. 13A, a lattice structure 290 can comprise a single lattice having two drooping sections, 292 and 294, and a third section 296. The two drooping sections 292 and 294 can be angled to have a dog-legged shape. The single lattice is secured at a proximal end to a proximal hub 300 and secured at a distal end to a distal hub 302. The outer section 292 at least partially encompasses an intermediate section 294 and the intermediate section 294 at least partially encompasses an inner section 296. The outer section 292 can define a proximal portion of the lattice structure 290, while all three sections can define a distal portion of the lattice structure 290. FIG. 13B is a schematic side view of the nested lattice structure 290 when slight tension is applied in opposite directions to the hubs 300 and 302 (i.e., stretched out).

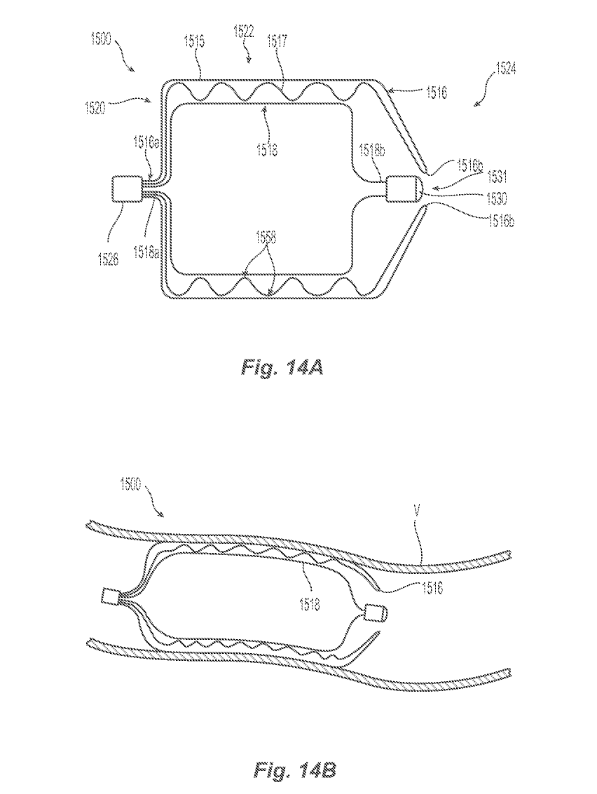

The lattice structure of the occlusion device can have one or more braided or mesh layers (collectively referred to herein as “braided” for ease of reference). Additionally, a single braided layer can include two or more sub-layers formed by everting the single braided layer to form a multi-layer construct within the single braided layer (as described above with regard to FIGS. 6A-6D). An everted braid comprising two or more sub-layers can comprise the innermost layers, intermediate layers and/or outermost layers of the lattice structure. For example, FIG. 14A shows one embodiment of an occlusion device 1500 having an everted outer occlusive braid 1516 that has an inner sub-layer 1517 and an outer sub-layer 1515. In the expanded configuration, the contact portion 1522 of the inner sub-layer 1517 can have an expanded, memory-set configuration with a corrugated contour while the outer sub-layer 1515 and the remaining portions of the inner sub-layer 1517 can have a contracted, memory-set configuration with a generally linear contour (as described in greater detail below).

In some embodiments, one or more layers of the lattice structure can individually have a free end or an open end that is not fixed to a hub. For example, FIG. 14A shows an occlusion device 1500 having an inner structural braid 1518 with proximal and distal ends 1518a, 1518b connected to the proximal and distal hubs 1526 and 1530, respectively, and an outer occlusive braid 1516 with a proximal end 1516a fixed to the proximal hub 1526 and a distal end 1516b defining an opening 1531 at a distal region 1524 of the device. The distal end 1516b is not fixed to a hub or other part of the device 1500 such that the distal end 1516b is free or otherwise unfixed. The distal hub 1528 moves independently of the distal end 1516b such that the occlusive and structural braids 1516 and 1518 can have different lengths without causing one of the braids to bunch upon collapse for delivery because the braids can move relative to each other to accommodate compression into a contracted state. FIG. 14B shows the occlusion device 1500 of FIG. 14A positioned within a vessel (V) or other body lumen. As shown, the outer occlusive braid 1516 can conform and seal to the inner anatomy of the vessel (V) independently of any radial compression or expansion of the structural braid 1518 as the blood vessel constricts and dilates.

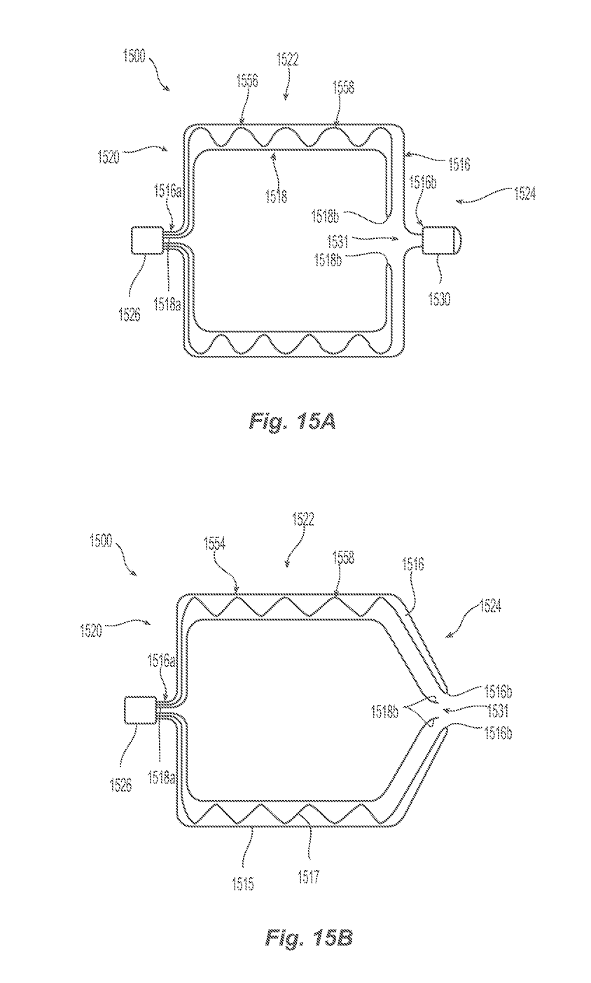

FIG. 15A shows another embodiment of an occlusion device 1500 having an outer occlusive braid 1516 with proximal and distal ends 1516a, 1516b connected to the proximal and distal hubs 1526 and 1530, respectively, and an inner structural braid 1518 with a proximal end 1518a fixed to the proximal hub 1526 and a free distal end 1518b defining an opening 1531 at a distal region 1524 of the device. FIG. 15B shows another embodiment of an occlusion device 1500 having only one hub 1526 disposed at the proximal region of the device 1500 that connects the proximal end 1516a of the occlusive braid 1516 and the proximal end 1518a of the structural braid 1518. As a result, the distal ends 1516b and 1518b are free floating members than can move radially and longitudinally with respect to the other braided layer. Likewise, an opening 1531 defines a distal-most region 1524 of the occlusion device 1500.

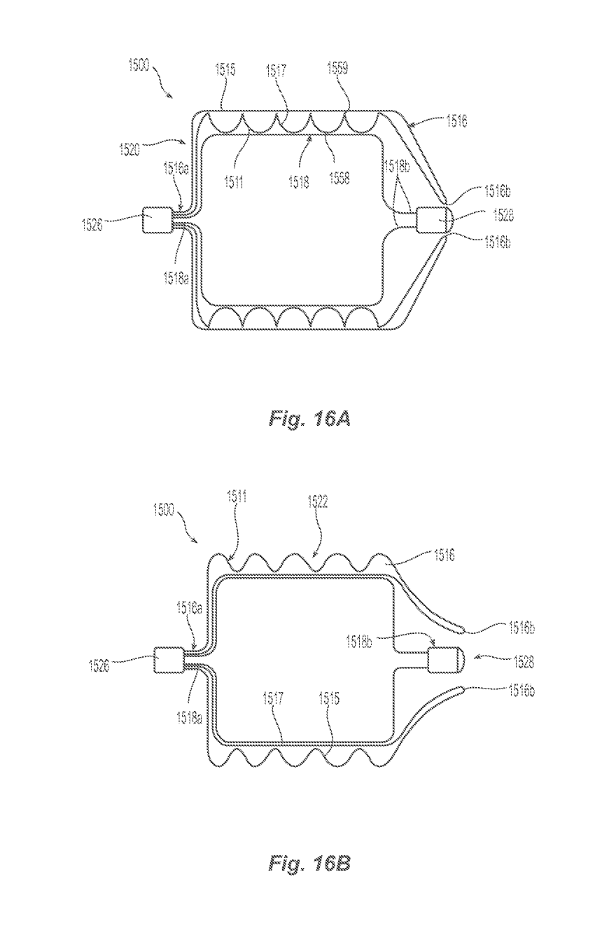

In some embodiments, the occlusion device can have corrugated portions (e.g., undulated, wave, saw-tooth, bellows-like, etc.) on one or more layers of the braids. The corrugated portions of the layer(s), for example, can have undulations with consistently smooth apices 1158 as shown in FIGS. 14A-15B or in other embodiments the corrugations can include one or more sharper and/or more distinct apices 1159 as shown in FIG. 16A. Additionally, the corrugated portion 1522 can define the outermost layer of the lattice structure (FIG. 16B), an intermediate layer (FIG. 16A), one or more sub-layers of an everted braid (FIGS. 16A-16B), and/or an innermost layer (not shown). In some embodiments, connecting sections 1511 between adjacent apices 1558 and/or 1159 of the corrugated portions can be generally linear as shown in FIG. 16B or saw-toothed as shown in FIG. 17A. In these and other embodiments, the connecting sections 1511 can have be exponentially-shaped (FIG. 16A).

In some embodiments, the corrugated portions of the occlusion device may comprise undulated, wave, saw-tooth, or bellows-like portions such that the apices 1558/1559 of the undulations touch or nearly touch adjacent portions of another braided layer and/or the same layer (e.g., an inner sub-layer touching an outer sub-layer) to form a plurality of substantially closed ring volumes. For example, FIG. 17A shows one embodiment of an occlusion device 1500 having first substantially closed ring volumes 1551 (“first volumes 1551”) between the inner sub-layer 1517 and outer sub-layer 1515 of the outer occlusive braid 1516, and second substantially closed ring volumes 1552 (“second volumes 1552”) between by the inner structural braid 1518 and the inner sub-layer 1517 of the occlusive braid 1516. As a result, at least a portion of the contact region 1522 of the occlusion device includes one or more baffles 1560 surrounding the first and/or second volumes 1551, 1552 and configured to trap emboli.

FIG. 17C is a cross-sectional side view of another embodiment of an occlusion device 1800 having an outer occlusive braid 1816 with an undulated portion 1822 that forms a ringed-pocket 1821 around a central portion of the occlusion device 1800. Similar to FIGS. 17A-17B, the ringed pocket 1821 can serve as a baffle-like portion of the device.

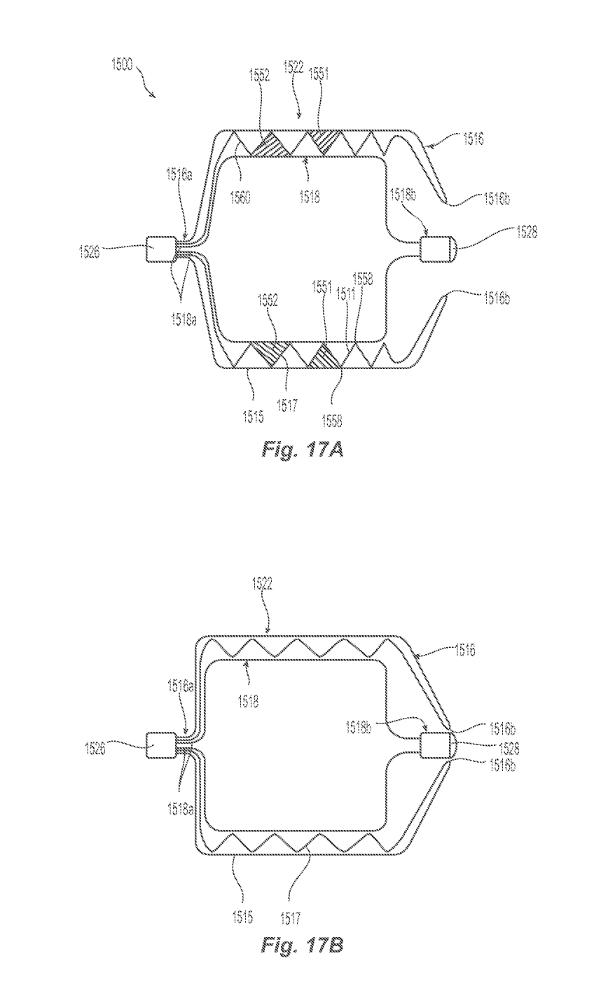

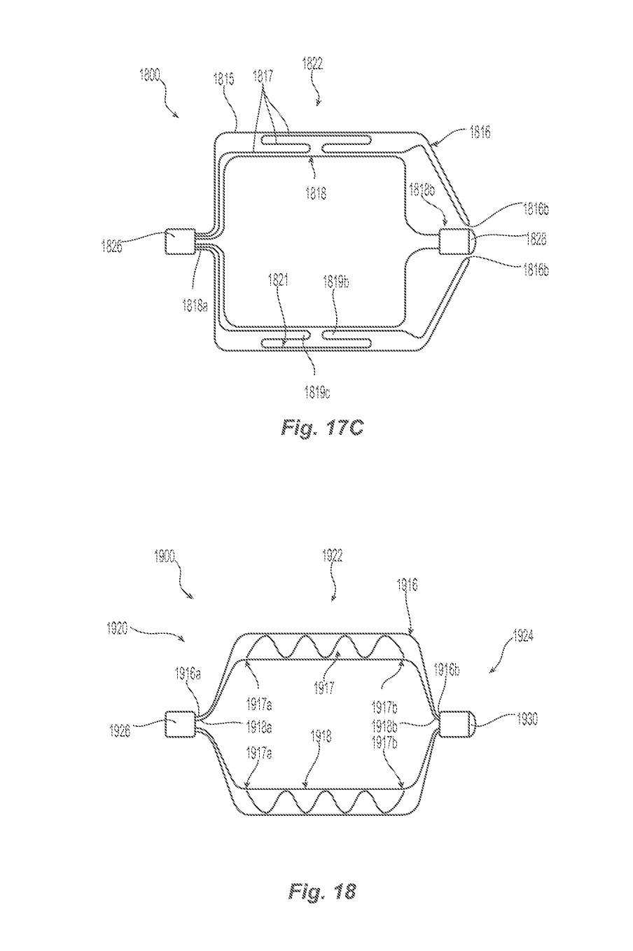

FIG. 18 shows yet another embodiment of an occlusion device 1900 having an outer occlusive braid 1916, an inner structural braid 1918, and an undulated intermediate braid 1917 sandwiched between the occlusive braid 1916 and the structural braid 1918. The intermediate braid 1917 can be a structural braid and/or an occlusive braid separate from the occlusive braid 1916 and inner structural braid 1918. The proximal ends 1916a, 1918a of the occlusive and structural braids, respectively, can be coupled to a proximal hub 1926 while the distal ends 1916b, 1918b of the occlusive and structural braids, respectively, can be coupled to a distal hub 1930. The intermediate braid 1917 has proximal ends 1917a positioned at a proximal portion of the contact region 1922 and distal ends 1917b positioned at a distal portion of the contact region 1922. In some embodiments the intermediate braid 1917 can be slidably positioned between the outer occlusive braid 1916 and inner structural braids 1918, or in other embodiments at least a portion of the intermediate braid can be coupled to the occlusive braid 1916 and/or structural braid 1918. For example, the proximal and distal ends 1917a, 1917b can be coupled to one or more braided layers of the lattice structure while the remaining length of the intermediate layer 1917 can be free to move within the space between the one or more braided layers. Additionally, the outer occlusive braid 1916 and the inner structural braid 1918 can be “as-braided” while the intermediate layer 1917 can be memory-set to expand to a desired configuration.

FIGS. 19A-19D show a process for making multi-layered lattice structures comprising both “as-braided” and memory-set (e.g., heat set, preset, etc.) braided layers and/or portions of braided layers. As used herein, “as-braided” refers to the state and/or configuration of the braid at the conclusion of fabrication on the mandrel 160 and before any heat and/or memory-setting treatments. Desired braid contours and/or shapes, such as corrugated portions, can be achieved by partial heat setting. As used herein, “partial heat setting” refers to the method by which portions of a single braid 1902 are heat set in a desired expanded configuration while other portions of the same braid forego any heat treatment. As a result, a braid can have one or more memory-set region(s) 1908 with memory-set expanded configurations and one or more “as-braided” region(s) 1906 that do not have expanded memory-set configurations. To begin the process, a braid 1902 having a first end 1904b and a second end 1904a is mounted on a mandrel 1900 (e.g., a mold) in an “as-braided” configuration (FIG. 19A). Next, the desired memory-set region(s) 1908 are selectively exposed to the heat setting process described above with reference to FIGS. 11A-11D, thereby molding the memory-set region 1908 of the braid to a desired expanded memory-set configuration (FIG. 19B). The “as-braided” regions 1906 are not subject to the same heat during the heat setting process. In some embodiments, the braid 1902 can have more than one memory-set region 1908 (e.g., two, three, four, etc.) along its length L and/or height H. Individual memory-set regions 1908 can have the same and/or different contours. Likewise, the braid 1902 can have more than one “as-braided” regions 1906 along its length L and/or height H.

Once the heat setting process is complete, the second end 1904a of the braid 1902 can be folded back towards the first end 1904b to create an inner layer 17 and an outer layer 15, as shown in FIG. 19C. The cross-sectional side view of FIG. 19D shows the braid 1902 once the second end 1904a have been pulled backwards far enough to generally line up with the first end 1904b. As shown, the resulting braid 1902 has an outer layer 15 defined by the “as-braided” region 1906 and an inner layer 17 comprising both “as-braided” regions 1906 and an undulating memory-set region 1908. In some embodiments, the braid can include a polymeric material along the “as-braided” region 1906 and a metal along the memory-set region(s) 1908.

FIGS. 20A-20E show a process for making multi-layered lattice structures comprising both expanded memory-set and contracted memory-set braided layers and/or portions of braided layers. Desired braid contours and/or shapes can be achieved by portioned heat setting. As used herein, “portioned heat setting” refers to the method by which portions of a single braid 1902 are heat set in a desired expanded configuration while other portions of the same braid are heat set in a desired contracted configuration. As a result, the braid 1902 can have one or more memory-set contracted region(s) 1902^{C} and one or more memory-set expanded region(s) 1902^{E}. To begin the process, a first portion 1920 of the braid 1902 is mounted on or in a first mandrel 1912 (e.g., a mold) that forces the first portion 1920 of the braid from an “as-braided” configuration into a desired expanded configuration (FIG. 20C). Heat can then be applied (as described above) to the first portion 1920 in the expanded configuration (FIG. 20D). The second portion 1930 of the braid 1902 is mounted on or in a second mandrel 1914 (or another portion of the first mandrel 1912 having a different shape) that forces the second portion 1930 of the braid from an “as-braided” configuration to a desired contracted configuration. For example, second portion 1930 can be placed over a second mandrel 1914 that is a tube having an outer diameter that is smaller than the fabrication mandrel 160 diameter and generally the same as the inner diameter of the delivery catheter. The second portion 1930 and any other subsequent portion can be molded and/or memory-set generally at the same time as the first portion 1920 or at a time after the first portion 1920. Additionally, the first mandrel 1912 and the second mandrel 1914 can be two portions of the same, contiguous mandrel. In some embodiments, the first and/or second portions 1920, 1930 can be secured to the first and/or second mandrels by one or more collars 1916. In some embodiments, the braid 1902 can have more than one expanded memory-set regions 1902^{E} (e.g., two, three, four, etc.) and/or contracted memory-set regions 1902^{C} along its length L and/or height H. Individual memory-set regions 1902^{C} and/or 1902^{E} can have the same and/or different contours.

Once the heat setting process is complete, the second end 1904a of the braid 1902 can be folded back towards the first end 1904b to create an inner layer 17 and an outer layer 15, as shown in FIG. 20D. The cross-sectional side view of FIG. 20E shows the braid 1902 once the second ends 1904a have been pulled backwards far enough to generally line up with the first ends 1904b. As shown, the resulting braid 1902 has an outer layer 15 defined by the contracted memory-set region 1902^{C} and an inner layer 17 defined by the expanded memory-set region 1902^{E}.

The processes and/or embodiments described above with reference to FIGS. 19A-20E can be applied to any embodiment of the occlusion device, lattice structure, occlusive braid and/or structural braid described herein.

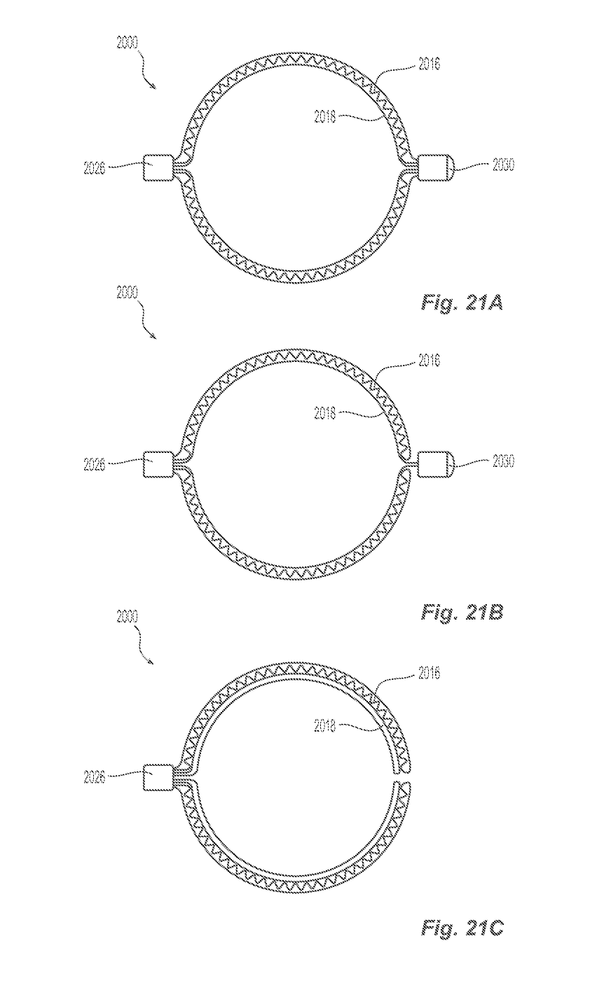

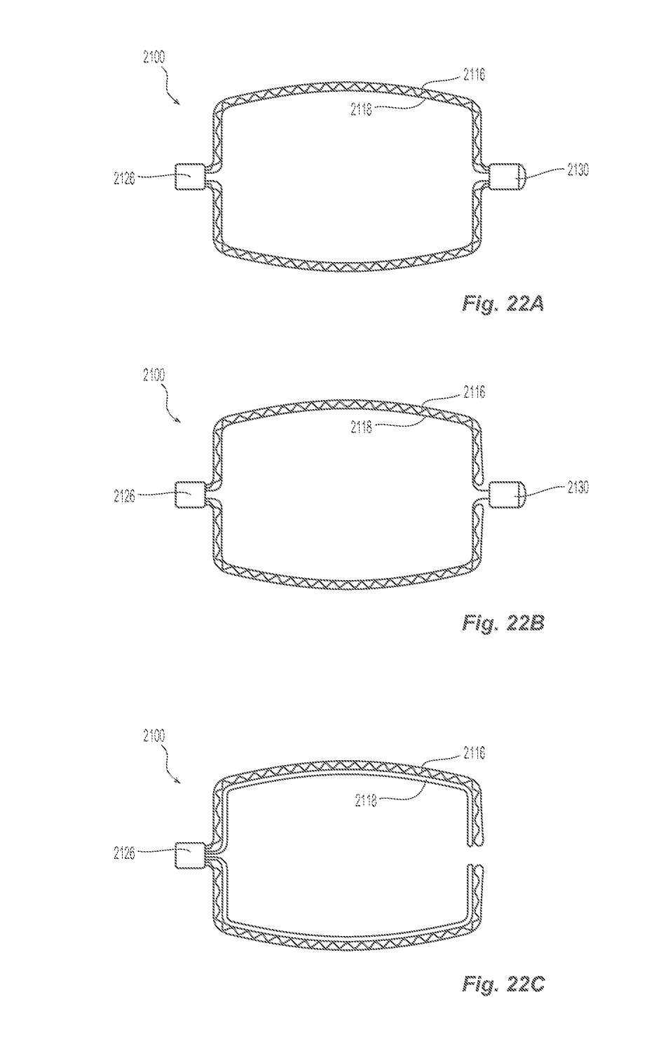

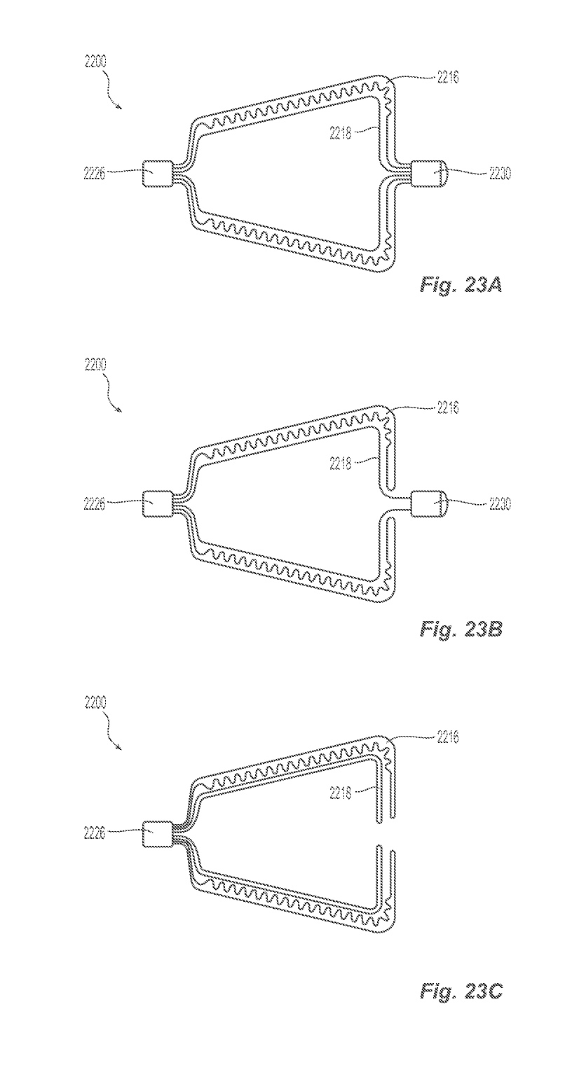

The occlusion device can have various geometries depending on the application. In some embodiments, an occlusion device can include one or more braided layers of the same lattice material or different lattice materials that have a generally cylindrical, spherical, ellipsoidal, oval, barrel-like, conical, frustum or other geometric shape. As described above, the braided layers or portions of the braided layers can have an undulated or wave-like contour, a saw-toothed contour, a bellows-like contour, a sinusoidal contour, and/or other suitable surface contours. For example, FIGS. 21A-21C show various embodiments of an occlusion device 2000 having a generally spherical shape. FIGS. 22A-22C show various embodiments of an occlusion device 2100 having a generally barrel-like shape. FIGS. 23A-23C show various embodiments of an occlusion device 2200 having a generally frustum-like shape.

It will be appreciated that specific elements, substructures, advantages, uses, and/or other features of the embodiments described with reference to FIGS. 12A-23C can be suitably interchanged, substituted or otherwise configured with one another and/or with the embodiments described with reference to FIGS. 6A-10D in accordance with additional embodiments of the present technology. For example, although the lattice structure of FIG. 12C is shown having mesh connectors 218 and 220, the spring coupling 256 from FIG. 12E may be substituted for mesh connectors 218 and 220. Furthermore, suitable elements of the embodiments described with reference to FIGS. 12A-23C can be used as standalone and/or self-contained devices.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A device for occluding a vascular structure, wherein the vascular structure has a first end and a second end and wherein at least the first end is exposed to blood flow, the device comprising:
an expandable lattice structure having a proximal region configured to be positioned at or near the first end, a distal region configured to extend into an interior portion of the vascular structure, and a contact region therebetween, wherein the expandable lattice structure includes—
an occlusive braid configured to contact tissue of the vascular structure and obstruct blood flow therethrough; and
a structural braid enveloped by the occlusive braid and coupled to the occlusive braid at a proximal hub located at the proximal region of the lattice structure;
wherein the structural braid is configured to drive the occlusive braid radially outward to press the occlusive braid against the tissue of the vascular structure at and/or distal to the first end; and
wherein at least one of the structural braid and the occlusive braid has an expanded memory-set region and a contracted memory-set region.

2. The device of claim 1 wherein the occlusive braid further comprises an outer layer and an inner layer, and wherein the outer layer has a contracted memory-set configuration and the inner layer has an expanded memory-set configuration.

3. The device of claim 1 wherein the occlusive braid has a contracted memory-set configuration and the structural braid has an expanded memory-set configuration.

4. The device of claim 1 wherein the structural braid has a contracted memory-set configuration and the occlusive braid has an expanded memory-set configuration.

5. The device of claim 1 wherein the distal region has a conical shape, and wherein the distal region has—
a first diameter at a proximal portion of the distal region;
a second diameter at a distal portion of the distal region, wherein the second diameter is smaller than the first diameter.

6. The device of claim 3 wherein the distal region is conically-shaped.

7. The device of claim 3 wherein the distal region further includes an atraumatic fastening member at a distal tip.

8. The device of claim 3 wherein the distal region of the occlusion device in the expanded configuration is defined only by the occlusive braid.

9. The device of claim 1 wherein the occlusive braid obstructs at least 95% of blood flow.

10. A device for occluding a vascular structure, wherein the vascular structure has a first end and a second end and wherein at least the first end is exposed to blood flow, the device comprising:
an expandable lattice structure having a proximal region configured to be positioned at or near the first end, a distal region configured to extend into an interior portion of the vascular structure, and a contact region therebetween, wherein the expandable lattice structure includes—
an occlusive braid configured to contact and seal with tissue of the vascular structure; and
a structural braid enveloped by the occlusive braid and coupled to the occlusive braid at a proximal hub located at the proximal region of the lattice structure;
wherein the structural braid is configured to drive the occlusive braid radially outward to press the occlusive braid against the tissue of the vascular structure at and/or distal to the first end; and
wherein at least one of the structural braid and the occlusive braid has a memory-set shaped portion and another portion with an as-braided configuration.

11. The device of claim 10 wherein:
the occlusive braid has a first proximal region, a first distal region and a first contact region in therebetween;
the structural braid has a second proximal region, a second distal region and a second contact region therebetween;
wherein at least one of the first contact region and the second contact region has a memory-set expanded configuration, and the first and second proximal regions and the first and second distal regions have an “as-braided” configuration when deployed.

12. The device of claim 10 wherein the occlusive braid comprises an outer layer and an inner layer.

13. The device of claim 12 wherein at least a portion of at least one of the outer layer and the inner layer has an “as-braided” configuration when deployed.

14. The device of claim 12 wherein:
the outer layer has a first proximal region, a first distal region and a first contact region therebetween;
the inner layer has a second proximal region, a second distal region, and a second contact region therebetween; and
wherein the only the first contact region has a memory-set expanded configuration.

15. The device of claim 14 wherein the memory-set expanded configuration has an undulating shape.

16. The device of claim 12 wherein:
the outer layer has a first proximal region, a first distal region and a first contact region therebetween;
the inner layer has a second proximal region, a second distal region, and a second contact region therebetween; and
wherein the only the second contact region has a memory-set expanded configuration.

17. The device of claim 16 wherein the memory-set expanded configuration has an undulating shape.

18. The device of claim 12 wherein:
the outer layer has a first proximal region, a first distal region and a first contact region therebetween;
the inner layer has a second proximal region, a second distal region, and a second contact region therebetween; and
wherein the only the first and second contact regions have memory-set expanded configurations.
